(19) United States
(12) Patent Application Publication
(10) Pub. No.: US 2018/0153841 A1
(43) Pub. Date: Jun. 7, 2018

(54) COMPOSITIONS AND METHODS FOR INDUCTION OF REMISSION OF INFLAMMATORY BOWEL DISEASES

(71) Applicant: KALMARNA LIMITED, ROAD TOWN (VG)

(72) Inventors: Alon ROSENBERG, REHOVOT (IL); Abraham MILSTEIN, REHOVOT (IL); Anthony MACKLE, MOY (GB); Ava Marie FIRTH, NOTTINGHAM (GB); Monique Michele SCHWARTZ, CLEARWATER, FL (US); Simon VAN DALSEM, ALKMAAR (NL); Arie HALPERN, PULLY (CH)

(73) Assignee: KALMARNA LIMITED, ROAD TOWN (VG)

(21) Appl. No.: 15/877,750

(22) Filed: Jan. 24, 2018

Related U.S. Application Data
(63) Continuation-in-part of application No. 15/527,724, filed on May 18, 2017.
(60) Provisional application No. 62/081,588, filed on Nov. 19, 2014.

(51) Int. Cl. A61K 31/198; A61K 9/00; A61K 47/26; A61K 47/12; A61K 47/18; A61P 1/00; A23L 33/125; A23K 50/30; A23K 50/10

(52) U.S. Cl. CPC A61K 31/198; A61K 9/0053; A61K 47/26; A61K 47/12; A23V 2002/00; A61P 1/00; A23L 33/125; A23K 50/30; A23K 50/10; A61K 47/183

(57) ABSTRACT

The present invention discloses compositions which comprises steviol glycoside, citric acid monohydrate, monosodium glutamate and/or glycine. Furthermore, the invention provides diets, nutraceuticals and nutrition oral rehydration therapy, means and methods useful in treating and inducing of remission of inflammation in IBD patients.

### CROSS REFERENCE TO RELATED APPLICATIONS

This application is a Continuation-in-Part of application Ser. No. 15/527,724, filed May 18, 2017, which is a U.S. National Stage of PCT International Application No. PCT/IB2015/058699, filed 11 Nov. 2015, which claims priority from provisional application No. 62/081,588, filed on Nov. 19, 2014. All of these applications are hereby incorporated by reference in their entirety.

### FIELD OF THE INVENTION

The invention relates to a composition and method for either treating or inducing of remission of IBD. More specifically, the composition offers compositions, diets, nutraceuticals and nutrition (e.g., EN, PEN), oral rehydration therapy, means and methods for treating or inducing of remission of inflammation in IBD patients.

### BACKGROUND OF THE INVENTION

Inflammatory bowel diseases (IBD), referring in this invention to Crohn's disease (CD), ulcerative colitis (UC) and related indications, such as irritable bowel syndrome (IBS), is a chronic immunologically mediated disease at the intersection of complex interactions between genetics, environment and gut microbiota. Signs and symptoms that are common to IBD and ulcerative colitis include: diarrhea, fever and fatigue, abdominal pain and cramping, blood in stool, reduced appetite, unintended weight loss etc. The exact cause of IBD is unclear but has been attributed to complex genetic and environmental factors that interact to trigger abnormal immune responses to commensal gut microbes. Because commensal microbes are thought to play a pivotal role in the development of IBD, there is considerable interest in targeting the commensal microbiome for therapeutic purposes. Diet, in particular, has emerged as an important lifestyle factor that greatly influences microbiome composition and regular dietary patterns are associated with IBD risk. Strict regulation of dietary intake has been shown to induce and, to some degree, maintain remission (i.e., symptom-free disease) in both children and adults with IBD, see MacLellan, Amber, et al. “The Impact of Exclusive Enteral Nutrition (EEN) on the Gut Microbiome in Crohn's Disease: A Review.” Nutrients 9.5 (2017): 0447 which is incorporated herein as a reference. It was underlined in literature that all current therapies are aimed at the downstream events, namely intervention directed towards the host inflammatory response. An alternative environmental factor, which has not been adequately explored in human subjects, is the effect of diet on IBD. Diet has an effect on the composition of the intestinal microbiome and gut immune status. Levine and others have proposed that CD may arise from a sequence of events involving changes in the microbiome, intestinal permeability leading to bacterial adherence or penetration of the epithelium, and subsequent stimulation of the adaptive immune response leading to tissue damage, see patent application WO2008004224 which is incorporated herein as a reference. Exclusive enteral nutrition (EEN) is a well-documented method of treatment. It involves placing children on a strict diet composed only of a single polymeric formula, as the sole source of nutrition over 6 to 8 weeks. Use of this treatment method, early in the disease, results in clinical remission in 50% to 80% of children by week 8 with no additional pharmacological treatment. Previous studies and clinical experience have shown that partial enteral nutrition (PEN) with 50% of calories from a formula with free diet is ineffective in inducing complete remission or reducing acute phase reactants, suggesting that the effect of EEN appears to depend, at least in part, on exclusion of free diet. In addition, since the mechanism of response or the triggering foods are unknown, there is no evidence based follow on strategy, to prevent recurrence upon re-exposure to normal diet.

Historically, simple salt-and-sugar solutions have been used in humans (especially children) to treat dehydration caused by both bacterial and viral diarrhea. These solutions provide Oral Rehydration Therapy (ORT) and were first used on a wide scale in the India, Pakistan and Bangladesh wars of 1971 where ORT successfully reduced mortality from 40% to 3% in the refugee camps, see Mahalanabis D, Choudhuri A B, Bagchi N G et al. Oral Fluid Therapy of Cholera among Bangladesh Refugees. Johns Hopkins Med J 1973; 79:473-479 which is incorporated herein as a reference. This was accomplished by the simple act of having parents' spoon small amounts of ORT into the children's mouths, instead of using intravenous drips (which were not available in the situation). The initial ORT recipe developed in 1971 was subsequently adopted by the World Health Organization and became known as ‘WHO Juice’. There have been slight updates to electrolyte and osmolarity concentrations, but the WHO recipe is still recommended as a first-line treatment for diarrhea epidemics today.

Px by Tonisity Ltd is the composition of the present invention. It contains electrolytes in the concentrations recommended by the World Health Organisation. Veterinary medicine has used ORT products to treat diarrhea in most species for over 40 years, see Atia A N, Buchman A L. Oral rehydration solutions in non-cholera diarrhea: a review. Am J Gastroenterol Nature Publishing Group, 2009; 104:2596-604; quiz 2605; Potter T. Neonatal calf scour—diagnosis, prognosis and treatment options. Vet Times 2015:12-14. http://www.vetsonline.com/publications/veterinary-times/archives/n-45-01/neonatal-calf-scoura-a-diagnosis-prognosis-and-treatment-options.html. Retrieved Jun. 15 2015; Reineke E L, Walton K, Otto C M. Evaluation of an oral electrolyte solution for treatment of mild to moderate dehydration in dogs with hemorrhagic diarrhea. J Am Vet Med Assoc 2013; 243:851-857; and Vukavic' T, Došen R. Fluid, electrolyte and acid-base changes in newborn piglets with acute diarrhoea. Vet Glas 1993; 47:383-386, all are incorporated herein as a reference.

The use of ORT in piglets was first reported in 1980 using a solution of glucose and glycine, see Taylor B E, McClave S A, Martindale R G et al. Guidelines for the Provision and Assessment of Nutrition Support Therapy in the Adult Critically Ill Patient: Society of Critical Care Medicine (SCCM) and American Society for Parenteral and Enteral Nutrition (A.S.P.E.N.). Crit Care Med 2016; 44:390-438, which is incorporated herein as a reference. In that report, piglets were experimentally infected with enterotoxigenic E. coli or rotavirus and then given either a solution of glucose/glycine or plain water. The same researchers then performed a study of naturally occurring diarrhea in a farm setting, using the same ORT. In the experimental scenario, preweaning mortality was significantly reduced from 24% to 11.6% (P<0.05). Diarrhea and sub-clinical dehydration are an important cause of pre-weaning mortality. Px has been scientifically shown to reduce mortality and fall-behinds. Early enteral nutrition is crucial in nurturing the intestine, even in the face of disease. Studies in humans with various medical or surgical conditions have shown the benefit of providing the right nutrients to the intestine, early in the course of disease. Fasting in the face of intestinal disease or surgery is no longer recommended in human medical guidelines, see Bywater R J, Woode G N. Oral fluid replacement by a glucose glycine electrolyte formulation in E coli and rotavirus diarrhoea in pigs. Vet Rec 1980; 106:75-8; and McClave S A, Martindale R G, Rice T W et al. Feeding the critically ill patient. Crit Care Med 2014; 42:2600-10; all incorporated herein as references. Consensus recommendations now suggest that early enteral nutrition should be provided, and that it should be in a simple, easily digested form. Tonisity Px takes the next logical step—since pigs are used as a model for human medicine and the monogastric digestive system—and applies those principles to nutrition for even suckling pigs. Px contains protein and amino acids that are specifically chosen to support the function of the intestinal cells.

Palatability of any oral product is hugely important. Anything that is meant to be ingested should also be palatable, particularly for pigs. The pig's acute sense of smell and taste is well-known and has been well studied. Not all ORT solutions are palatable to pigs.

Px has a combination of flavours that pigs find highly appealing, which increases their intake of the product at the times when they need it most.

The fundamental principle of ORT is to supply simple sugars, electrolytes and water to restore hydration and glucose levels in the body. The small intestinal cells (enterocytes) can only absorb simple sugars such as glucose, fructose and galactose, which then enter the blood stream and are used for glycolysis. The transport of these simple sugars into the enterocyte relies upon Na—K-ATPase dependent transporters in the enterocyte cell membrane. These transporters are dependent on having baseline concentrations of sodium and potassium available to them in order to accomplish their task. When the intestine is under stress and the digestive processes are impaired, it is particularly important to provide these three things—sugar, sodium and potassium—so that enterocytes can do their work most efficiently and so that water can be absorbed.

ORT solutions generally contain monosaccharides (usually glucose) and electrolytes. ORT solutions are also usually isotonic, with an osmolarity of about 270-300 mOsm/l. In its basic formulation, Px would qualify as a strong ORT product because it is appropriately balanced AND has good palatability to swine. However, Px is unique because it also contains certain proprietary ingredients that are formulated to support the metabolic functions of the enterocytes themselves.

### SUMMARY OF THE INVENTION

The invention hence discloses a novel and effective composition (interchangeably denoted hereinafter in the terms “Px” or “Tonicity” and the “composition”, provided useful for either treating IBD or inducing of remission of IBD patients, and characterized by that that the composition consists at least two member of a group consisting of steviol glycoside, citric acid monohydrate, monosodium glutamate and glycine. Additionally or alternatively, this composition characterized by that that the composition consists at least three member of a group consisting of steviol glycoside, citric acid monohydrate, monosodium glutamate and glycine. Additionally or alternatively, this composition is characterized by that that said composition consisting of steviol glycoside, citric acid monohydrate, monosodium glutamate and glycine.

The invention also discloses a composition as defined in any of the above, wherein the ingredients are (i) about 85% steviol glycoside extract ranging from about 0.01% to about 0.03% w/w, (ii) about 0.20% w/w citric acid monohydrate; (iii) monosodium glutamate ranging from about 0.05% to about 0.80% w/w; and (iv) about 0.35% w/w glycine.

The invention also discloses a composition as defined in any of the above, wherein the at least one of the following is held true: (i) the composition comprises 1-glutamic acid in a range of about 0.01% to about 0.40% w/w and monosodium glutamate in a range of about 0.05% to about 0.80% w/w; (ii) the composition comprises about 1.50% w/w glucose monohydrate; (iii) the composition comprises about 0.20% w/w sodium chloride; (iv) the composition comprises about 0.15% w/w potassium chloride; (v) the composition comprises about 0.15% w/w sodium dihydrogen phosphate; (vi) the composition comprises about 0.10% w/w xanthan gum; (vii) the composition comprises about 0.35% w/w glycine; (viii) the composition comprises about 0.30% w/w trisodiumcitrate; (ix) the composition comprises about 0.20% w/w citric acid monohydrate; (x) the composition comprises 85% Steviol Glycoside extract in a range of about 0.01% to about 0.03% w/w; (xi) wherein the composition comprises hydrolyzed whey in a range of about 0.15% to about 1.00% w/w; (xii) the composition comprises about 1.00% w/w hydrolyzed wheat; (xiii) the composition comprises cereals as a protein source.

The invention also discloses a composition as defined in any of the above, wherein it further comprises at least one or more members of a group consisting of 1-glutamic acid in a range of about 0.01% to about 0.40% w/w; about 1.50% w/w glucose monohydrate; about 0.20% w/w sodium chloride; about 0.15% w/w potassium chloride; about 0.15% w/w sodium dihydrogen phosphate; about 0.10% w/w xanthan gum; and hydrolyzed whey in a range of about 0.15% to about 1.00% w/w.

The invention also discloses a composition as defined in any of the above, wherein aforesaid at least one composition is (i) tailored to suit the flavouring preferences of different species or individuals; (ii) a ready-to-use composition; (iii) is a powder concentrate, or the concentrate is diluted in water; (iv) it comprises enzyme co-factors; and/or (v) it comprises a monosaccharide.

The invention also discloses a composition as defined in any of the above, wherein the composition is an isotonic, hypotonic or hypertonic solution. It is in the scope of the invention wherein the isotonic solution is comparable to the isotonicity of a 0.9% solution of sodium chloride.

The invention also discloses a composition as defined in any of the above, wherein the composition is formulated or administrated as a gel, a spray or a quick dissolve tablet.

The invention also discloses a composition as defined in any of the above, wherein the composition is provided in one member of a group consisting of diets, foodstuffs, beverages, top-dressings, food dressings, food additives, drugs, medicaments, pharmaceuticals, nutraceuticals and nutrition, including EN and PEN, and oral rehydration therapy.

The invention also discloses a composition as defined in any of the above, wherein the composition is provided in one member of a group consisting of diets, foodstuffs, edibles beverages, top-dressings, food dressings, food additives, drugs, medicaments, pharmaceuticals, nutraceuticals and nutrition, including EN and PEN, carriers for nutraceuticals, medicament (including oral vaccine & antibiotics) & flavour enhancers [NW's] prebiotics and toppers.

The invention also discloses a method for either treating IBD or inducing of remission of IBD patients, characterized by administering a composition consists at least two member of a group consisting of steviol glycoside, citric acid monohydrate, monosodium glutamate and glycine.

The invention also discloses a method for either treating IBD or inducing of remission of IBD patients, characterized by administering a composition consists at least three member of a group consisting of steviol glycoside, citric acid monohydrate, monosodium glutamate and glycine.

The invention also discloses a method for either treating IBD or inducing of remission of IBD patients, characterized by administering composition consisting of steviol glycoside, citric acid monohydrate, monosodium glutamate and glycine.

The invention also discloses a method for either treating IBD or inducing of remission of IBD patients, characterized by administering a composition comprising (i) about 85% steviol glycoside extract ranging from about 0.01% to about 0.03% w/w, (ii) about 0.20% w/w citric acid monohydrate; (iii) monosodium glutamate ranging from about 0.05% to about 0.80% w/w; and (iv) about 0.35% w/w glycine.

The invention also discloses a method for either treating IBD or inducing of remission of IBD patients, wherein at least one of the following is held true: (i) the composition comprises 1-glutamic acid in a range of about 0.01% to about 0.40% w/w and monosodium glutamate in a range of about 0.05% to about 0.80% w/w; (ii) the composition comprises about 1.50% w/w glucose monohydrate; (iii) the composition comprises about 0.20% w/w sodium chloride; (iv) the composition comprises about 0.15% w/w potassium chloride; (v) the composition comprises about 0.15% w/w sodium dihydrogen phosphate; (vi) the composition comprises about 0.10% w/w xanthan gum; (vii) the composition comprises about 0.35% w/w glycine; (viii) the composition comprises about 0.30% w/w trisodiumcitrate; (ix) the composition comprises about 0.20% w/w citric acid monohydrate; (x) the composition comprises 85% Steviol Glycoside extract in a range of about 0.01% to about 0.03% w/w; (xi) wherein the composition comprises hydrolyzed whey in a range of about 0.15% to about 1.00% w/w; (xii) the composition comprises about 1.00% w/w hydrolyzed wheat; (xiii) the composition comprises cereals as a protein source.

The invention also discloses a method for providing anoral rehydration therapy, improving small intestine anatomy & increasing small intestine's villi size weight gaining, weight gaining associated with reduction in antibiotics usage, for improving weight gain whilst decreasing use of milk replacers, improving small intestine anatomy & increasing small intestine's villi size, or increasing the uptake of nutraceuticals, medicament (including oral vaccine & antibiotics) & flavour enhancers [NW's] uptake, comprising step of administering a patient a composition defined in any of the above.

### BRIEF DESCRIPTION OF THE FIGURES

The present invention will be readily understood by the following detailed description in conjunction with the accompanying drawings and in which:

FIG. 1 illustrates Average Daily Consumption of Px by Litters (n=58) according an embodiment of the invention;

FIG. 2 illustrates Daily DMI from 1 to 3 Days before weaning (g/pig/day) according to another embodiment of the invention;

FIG. 3 illustrates Daily DMI intake for 6 days Post-Weaning (g/pig/day) according to another embodiment of the invention;

FIG. 4 illustrates Gruel Intake Comparison according to another embodiment of the invention;

FIG. 5 illustrates Villi from Control Group (Jejunum) according to another embodiment of the invention;

FIG. 6 illustrates Villi from Px Group (Jejunum) according to another embodiment of the invention;

FIG. 7 illustrates Comparison of Px v Control in Scouring Piglets according to another embodiment of the invention;

FIGS. 8a & 8b illustrate Piglets Body Weight (kg) on Day 6 according to another embodiment of the invention.

FIG. 9 illustrates Px Consumption by Litters;

FIG. 10 illustrates Weight Comparison at Day 19;

FIG. 11 illustrates Relative Risk of Pre-Weaning Mortality;

FIG. 12 illustrates Pre-Weaning Mortality in Trials;

FIG. 13 illustrates Summary of Study Design;

FIG. 14 illustrates Daily DMI from 1 to 3 Days Before Weaning;

FIG. 15 illustrates Daily DMI intake for 6 days Post-Weaning;

FIG. 16 illustrates Jejunum from Px Group Post-Weaning; and

FIG. 17 illustrates Jejunum from Control Group Post-Weaning.

### DETAILED DESCRIPTION OF THE PREFERRED EMBODIMENT

Px by Tonisity is the first isotonic protein drink that contains key ingredients to support the intestinal cells, the enterocytes. The basis of Px is to nourish the enterocyte. Enterocytes are the final gateway for the absorption of proteins and carbohydrates into the body. If the enterocytes are working efficiently, then the body is able to absorb more nutrients. Px contains ingredients which support the efficient function of the enterocytes.

By supporting the enterocytes, Px helps pigs overcome the key productivity hurdles of young pigs, namely pre-weaning mortality (PWM) and a smooth transition through weaning. There are many aspects to successful management of young pigs but these hurdles are always a challenge. Various strategies are used to try to overcome these hurdles. Milk replacer, electrolyte solutions, and quick-start drenches are all used by producers to give extra energy and fluids to piglets. Antibiotics, probiotics, pre-biotics and plant extracts are all used in an attempt to modify the intestinal bacteria population. None of those strategies actually improve the function of the intestinal cells (enterocytes), which are the engines that are responsible for absorbing any nutrients. A more focused approach is to nourish the intestinal cells themselves and help them to do the best job they can of absorbing nutrients and this is the approach that Tonisity has taken in the development of Px. Tonisity has taken great care to investigate how best to use Px and to demonstrate the return on investment to producers. Over the last two years, Tonisity has conducted over 27 trials involving over 760 litters and 9600 pigs on production farms in the USA, United Kingdom and Spain. Key findings from those trials are presented here. This research has shown that Px has a positive impact on pre-weaning mortality and post-weaning weight, as well as other key parameters.

### Oral Rehydration Therapy in Gastrointestinal Disease

While it might seem counter-intuitive to administer enteral products to patients who have gastroenteritis, this is exactly what was proven to work in human medicine. Gastroenteritis has many aetiologies in farm animals. Most are viral, though some patients develop gastroenteritis for unknown reasons. Dietary indiscretion is also reasonably common. Parvovirus is a particularly severe form of gastroenteritis in dogs. Cells lining the intestinal tract are directly attacked by parvovirus, causing inflammation, subnormal absorption of nutrients, and hemorrhages. Parvovirus also causes a particularly severe nausea and secretory diarrhea. Many veterinary staff are reluctant to feed patients who are vomiting, especially those with parvovirus. However, in a well-designed study of 30 parvovirus pups less than 24 weeks old, Mohr et al showed a more rapid return of appetite, more rapid weight gain and better intestinal wall integrity in pups who received early enteral nutrition by naso-oesophageal tube compared to those who were fed a low-fat tinned food (Mohr et al., Effect of early enteral nutrition on intestinal permeability, intestinal protein loss, and outcome in dogs with severe parvoviral enteritis. J Vet Intern Med. 2003 November-December; 17(6):791-8.—which is incorporated herein as a reference).

In another well-designed study on piglets, Kansagra et al. showed that the lack of enteral nutrition leads to gut atrophy, specifically mucosal atrophy. The study showed notable decreases in jejunal mass (34.8%), villus height (44.4%), and villus area (56.1%) of non-enteral-fed piglets compared with controls. However, in the ileum, only tissue mass (33.9%), protein, and DNA content were reduced by lack of enteral nutrition, whereas villus height and area were unaffected. These findings are not necessarily novel and highlight the fact that the proximal mucosa is more susceptible to lack of ENT nutrients than the distal gut (Kansagra et al., Total parenteral nutrition adversely affects gut barrier function in neonatal piglets. Am J Physiol Gastrointest Liver Physiol. 2003 December; 285(6):G1162-70—which is incorporated herein as a reference).

A third type of gastrointestinal problem that is common in small animal patients is the post-operative recuperation after gastrointestinal surgery. Again, historically, recommendations were to not feed the patient for at least 24 hours after surgery and sometimes longer. Patients who have recently undergone intestinal surgery are at particular risk of intestinal motility disorders, particularly ileus. However, the presence of food within the intestine actually promotes normal motility and stimulates mucosal perfusion, which speeds healing (Chan D L, Gastrointestinal dysfunction in the critical patient. 2007. British small animal veterinary association, Birmingham England—which is incorporated herein as a reference).

The oral liquid rehydration formulation of the present invention is ideal for treating the above conditions. Due to the presence of a flavouring which is palatable to animals, it is also more likely to be taken voluntarily by the ill animal, and due to the presence of glutamate-based specific amino acids.

Since the oral liquid rehydration formulation of the present invention is produced as concentrate powder and also as pre-mixed, it can easily be used in the farm setting for animals who are unwell, but not ill enough to require care in a clinic. It is equally useful for animals who are recuperating following care in a clinic or even human patients recovering at their homes.

The term ‘about’ refers hereinafter to a value being 25% lower or greater than the defined measure.

### Example 1

### Administering Oral Rehydration Therapy

Oral rehydration therapy fluid of the present invention may be initially given at a rate of about 0.5 ml/kg every 2 hours, given orally, using a syringe if necessary. This is a very small volume which rarely precipitates vomiting. If no vomiting occurs, this volume may be increased by 50% every 8-12 hours. For cats and small dogs, ice cube trays can be used to freeze small blocks of the oral liquid rehydration formulation and then dispensed as needed. Once the patient begins to lap the fluid, then volumes can be increased rapidly and more calorie-dense food can be introduced. Oral rehydration therapy also has a place in outpatient treatment of various other conditions. Oral rehydration therapy, particularly if it is a highly palatable product such as the oral liquid rehydration formulation of the present invention, can be used as part of a dietary management plan for patients with mild gastroenteritis. In many of these patients, a short period of fasting combined with small amounts of fluid intake is sufficient to relieve the symptoms. Owners and lay staff can easily learn how to administer the oral liquid rehydration formulation of the present invention, as no special skill is required to use it.

### Example 2

An example of a hydrolyzed wheat protein source oral rehydration formulation according to the invention is as follows:

| Ingredient | in % |
|---|---|
| Water | 96.27 |
| Glucose monohydrate | 1.50 |
| Sodium chloride | 0.26 |
| Potassium chloride | 0.15 |
| Glycine | 0.40 |
| Trisodiumcitrate | 0.29 |
| Xanthan gum | 0.05 |
| Hydrolysed wheat protein | 1.00 |
| L-glutamic acid | 0.04 |
| Monosodium glutamate | 0.04 |

Concentrate Formulation of Example 2. A concentrate may be prepared as follows:

| Ingredient | in % |
|---|---|
| Water | 62.70 |
| Glucose monohydrate | 15.00 |
| Sodium chloride | 2.60 |
| Potassium chloride | 1.50 |
| Glycine | 4.00 |
| Trisodiumcitrate | 2.90 |
| Xanthan gum | 0.50 |
| Hydrolysed wheat protein | 10.00 |
| L-glutamic acid | 0.40 |
| Monosodium glutamate | 0.40 |

The concentrate is mixed and held at room temperature for at least 5 minutes. Typical pH is about 3.7 to 3.8 at 20° C. after 10× dilution, for use. To dilute for use, 10 ml of concentrate is added into 90 ml water and mixed.

### Example 3

### Oral Liquid Rehydration Formulation for Pigs, Calves and Lambs

An example of a hydrolized wheat protein source oral rehydration formulation according to the invention is as follows:

| Ingredient | in % |
|---|---|
| Water | 96.26 |
| Glucose monohydrate | 1.50 |
| Sodium chloride | 0.26 |
| Potassium chloride | 0.15 |
| Glycine | 0.40 |
| Sodium dihydrogen phosphate | 0.10 |
| Xanthan gum | 0.05 |
| Hydrolysed wheat protein | 1.00 |
| L-glutamic acid | 0.04 |
| Monosodium glutamate | 0.04 |

Concentrate Formulation of Example 3. A concentrate may be prepared as follows:

| Ingredient | in % |
|---|---|
| Water | 62.60 |
| Glucose monohydrate | 15.00 |
| Sodium chloride | 2.60 |
| Potassium chloride | 1.50 |
| Glycine | 4.00 |
| Sodium dihydrogen phosphate | 1.00 |
| Xanthan gum | 0.50 |
| Hydrolysed wheat protein | 10.00 |
| L-glutamic acid | 0.40 |
| Monosodium glutamate | 0.40 |

The concentrate is mixed and held at room temperature for at least 5 minutes. Typical pH is about 3.7 to 3.8 at 20° C. after 10× dilution, for use. To dilute for use, 10 ml of concentrate is added into 90 ml water and mixed.

### Example 4

### Oral Liquid Rehydration Formulation for Pigs, Calves and Lambs

An example of a hydrolized wheat protein source oral rehydration formulation according to the invention is as follows:

| Ingredient | in % |
|---|---|
| Water | 97.00 |
| Glucose monohydrate | 1.45 |
| Sodium chloride | 0.26 |
| Potassium chloride | 0.15 |
| Glycine | 0.30 |
| Sodium dihydrogen phosphate | 0.10 |
| Xanthan gum | 0.10 |
| Citric Acid Monohydrate | 0.20 |
| Hydrolysed whey protein | 0.15 |
| L-glutamic acid | 0.04 |
| Monosodium glutamate | 0.25 |
| 85% Steviol Glycoside extract | 0.001 |

Powder Formulation of Example 4. A powder may be prepared as follows:

| Ingredient | in % |
|---|---|
| Water | 97.00 |
| Glucose monohydrate | 48.33 |
| Sodium chloride | 8.67 |
| Potassium chloride | 5.00 |
| Glycine | 10.00 |
| Sodium dihydrogen phosphate | 3.33 |
| Xanthan gum | 3.33 |
| Citric Acid Monohydrate | 6.67 |
| Hydrolysed whey protein | 5.00 |
| L-glutamic acid | 1.33 |
| Monosodium glutamate | 8.30 |
| 85% Steviol Glycoside extract | 0.033 |

The powder is mixed and held at room temperature for at least 5 minutes. Typical pH is about 3.8 to 4.0 at 20° C. after dilution of 3.0 parts of powder with 97.0 parts of water. To dilute for use, 468 gr. of powder in 4 US gallons (15.14 liters) of water and mixed.

### Example 5

### Oral Liquid Rehydration Formulation for Pigs, Calves and Lambs

Another example of a hydrolized whey protein source oral rehydration formulation according to the invention is as follows:

| Ingredient | in % |
|---|---|
| Water | 97.00 |
| Glucose monohydrate | 1.44 |
| Sodium chloride | 0.26 |
| Potassium chloride | 0.15 |
| Glycine | 0.30 |
| Sodium dihydrogen phosphate | 0.10 |
| Xanthan gum | 0.10 |
| Citric Acid Monohydrate | 0.20 |
| Hydrolysed whey protein | 0.15 |
| L-glutamic acid | 0.04 |
| Monosodium glutamate | 0.25 |
| 85% Steviol Glycoside extract | 0.01 |

Powder Formulation of Example 5. A powder may be prepared as follows:

| Ingredient | in % |
|---|---|
| Water | 97.00 |
| Glucose monohydrate | 48.03 |
| Sodium chloride | 8.67 |
| Potassium chloride | 5.00 |
| Glycine | 10.00 |
| Sodium dihydrogen phosphate | 3.33 |
| Xanthan gum | 3.33 |
| Citric Acid Monohydrate | 6.67 |
| Hydrolysed whey protein | 5.00 |
| L-glutamic acid | 1.33 |
| Monosodium glutamate | 8.30 |
| 85% Steviol Glycoside extract | 0.33 |

The powder is mixed and held at room temperature for at least 5 minutes. Typical pH is about 3.8 to 4.0 at 20° C. after dilution of 3.0 parts of powder with 97.0 parts of water. To dilute for use, 468 gr. of powder in 4 US gallons (15.14 liters) of water and mixed.

### Set of Examples, Collectedly are Herein Referred as Experiment 6

The present invention also discloses isotonic oral compositions for nourishing IBD patients. The composition comprises ingredients selected from the group consisting of water, glucose monohydrate, sodium chloride, potassium chloride, glycine, sodium dihydrogen phosphate, xantham gum, hydrolysed whey protein, and wherein the compositions further consists steviol glycoside extract, 1-glutamic acid, monosodium glutamate, citric acid monohydrate and any combination thereof. Those compositions are provided useful in diets, top-dressing, foodstuffs, nutraceuticals and nutrition (e.g., EN, PEN), means and methods for treating or inducing of remission of inflammation in IBD patients.

Effect of Px when Given to Suckling Pigs

### Effect of Px on Pre-Weaning Mortality and Post-Weaning Weight (TON-USA-034)

The aim of this study was to determine the effect of Px on pre-weaning mortality and post-weaning weight when given to suckling pigs from days 2-8 of age.

### Materials and Methods

The study was conducted in a farrow-to-nursery operation with 7200 sows located in Minnesota, USA. The farm has an average live-born of 12.8 pigs and a historic pre-weaning mortality of 13-14%. The farm was negative for PRRS at the time of the trial. Seventy-three sows (PIC) and their litters (968 piglets) were enrolled in the study. Sows and their litters were randomised to one of two groups. Litters in the Px group received 500 mL of Px in an open pan, once daily from day 2-8 of life. Litters in the control group received nothing. All piglets had access to an automatic drinker. The amount of Px consumed was recorded daily. On day 2 of life, piglets were individually ear-tagged and weighed. Piglets were again weighed at day 8, at weaning (˜day 19) and at day 35. ADG at weaning was calculated based on actual days of age. Mortality and apparent cause of death were recorded daily. Body weight and average daily gain for each interval were analysed using a t-test with Welch's correction. Results were considered significant at P≤0.05 and considered a trend at P>0.05 and P≤0.10. Statistical analysis was performed using GraphPad Prism 7 software.

### Results

Three litters were withdrawn from the trial on day 2 due to sow or litter health issues.

### Pre-Weaning Mortality

The pre-weaning mortality was significantly lower in the Px group (10.3%) compared to the control group (15.0%) (P=0.029).

TABLE 1
Pre-weaning Mortality

| | Survived | Died | PWM |
|---|---|---|---|
| Control | 361 | 64 | 15.1% |
| Px | 454 | 52 | 10.3% |

### Weight Gain

There was no significant difference between the groups at day 2 (baseline). At day 8, the Px group had gained an average of 1.55 kg [3.4 lb], compared to control group that had gained an average of 1.25 kg [2.75 lb]. The difference in net gain between groups at day 8 was statistically significant at P=0.0002. Piglets were between 15-21 days of age when weaning weights were measured. No significant differences between groups were seen at weaning. By day 35, differences in body weight and ADG were significantly different (P=0.041) between groups, with the Px pigs weighing 9.8 kg [21.6 pounds] and the control pigs weighing 8.78 kg [19.3 pounds].

TABLE 2
Weight Gain

| | Control | Px | Signifi- |
|---|---|---|---|
| | (Mean ± SEM) | (Mean ± SEM) | cance |
| BW day 2 | 3.67 ± 0.05, | 3.72 ± 0.047, | 0.5123 |
| | n = 459 | n = 507 | |
| BW day 8 | 6.94 ± 0.12, | 7.16 ± 0.11, | 0.1715 |
| | n = 430 | n = 505 | |
| Net gain | 2.75 ± 0.14, | 3.43 ± 0.12, | 0.0002 |
| d 2 − d 8 | n = 473 | n = 470 | |
| BW day 18 | 12.54 ± 0.19, | 12.39 ± 0.17, | 0.5370 |
| | n = 405 | n = 454 | |
| Wt d 35 | 19.36 ± 0.67, | 21.61 ± 0.92, | 0.0491 |
| | n = 76 | n = 92 | |
| | (8.78 ± 0.30 kg) | (9.80 ± 0.41 kg) | |
| Net gain | 15.72 ± 0.61, | 17.73 ± 0.90, | 0.0660 |
| d 2 − d 35 | n = 76 | n = 92 | |
| ADG | 0.55 ± 0.019, | 0.62 ± 0.03, | 0.0491 |
| d 2 − d 35 | n = 76 | n = 92 | |
| | (0.250 ± 0.008 kg) | (0.280 kg ± 0.02 kg) | |

Note:
all weight and adg values in this table are in pounds except where indicated

### Conclusions and Relevance

Px supplementation in the first week of life reduced PWM by 33% in this trial. Px supplementation had a clear impact on weight gain in the first week of life, which was again seen at day 35. No clear effect was measured at the time of weaning, but the impact of Px was confirmed. at day 35

### Effect of Px on Suckling Pig Mortality and Weaning Weight

### Introduction

The aim of this study by Tonisity was to assess the impact of Px on pre-weaning mortality and weight gain in suckling pigs. A randomised, controlled prospective clinical trial was performed.

### Materials and Methods

One hundred twelve sows (Landrace×Large White or Danbred) and their litters (1496 piglets) from 2 farrowing batches were used in the study. The farm was located in central Spain, and was a farrow-to-nursery operation with 2500 sows with an average live-born of 13.1 pigs and a historic pre-weaning mortality of 13.7%. The farm has a history of scours during lactation with Clostridium difficile, Clostridium perfringens, Escherichia coli and type A rotavirus. Sows and their litters were randomised to one of two groups. Litters in the Px group received 500 mL of Px in an open pan, once daily from day 2-8 of life. Litters in the control group received nothing. All piglets had access to an automatic drinker. The amount of Px consumed was recorded daily. On the day of farrowing, piglets were individually ear-tagged and weighed. Piglets were again weighed at day 8 and day 19. Creep feed was started in all litters at day 10. Mortality and apparent cause of death were recorded daily.

### Results

Piglets in the Px group weighed more than the control group at day 8 (P<0.1) and 240 grams (0.53 lb) more at day 19 (P<0.05). Pigs in the Px group had a higher average daily gain both in the first week (P<0.1) and until day 19 (P<0.05). Day 19 mortality in the Px group was 5.2% compared to 6.7% in the control group, a reduction of 17% (P<0.001), though both groups had much lower mortality than was usual for the farm.

TABLE 3
Weight Gain and Mortality in Suckling Pigs

| | Px* | Control* | P value |
|---|---|---|---|
| Body weight d 1 | 1.41 ± 0.04 | 1.37 ± 0.04 | NS |
| (kg ± SE) | | | |
| Body weight d 8 | 2.29 ± 0.05 | 2.23 ± 0.05 | P < 0.1 |
| (kg ± SE) | | | |
| Body weight d 19 | 4.25 ± 0.11 | 4.01 ± 0.11 | P < 0.05 |
| (kg ± SE) | | | |
| ADG d 1 − d 8 (kg/d) | 0.123 ± 0.007 | 0.114 ± 0.007 | P < 0.1 |
| ADG d 1 − d 19 (kg/d) | 0.158 ± 0.006 | 0.145 ± 0.006 | P < 0.05 |
| Mortality d 8 (%) | 3.7 | 4.5 | P = 0.424 |
| Mortality d 19 (%) | 5.2 | 6.7 | P < 0.001 |

*Values are least-squares means ± standard error.
NS = not significantly different.
BOLD indicates statistically significant P value.

Consumption of Px increased steadily during the week 0. By day 8, virtually all litters were consuming all 500 mL of Px that they were offered. Litters contained an average of 14 piglets, equating to an average consumption of 36 mL/pig after day 3. Reference is made to FIG. 1, shoeing the average daily consumption of Px by Litters (n=58)

### Conclusions

This study demonstrated that Px had a beneficial effect on suckling piglets when given during the first week of life, and that these benefits were measurable at the time of weaning. Contrary to accepted practice, piglets less than one week old will drink significant volumes of Px. Statistically significant differences that are meaningful to actual production were seen in body weight, average daily gain and pre-weaning mortality (PWM). The control group's pre-weaning mortality in this study (6.7%) was lower than normal due to the extra care and attention being given to the piglets by the study personnel. Nevertheless, the pre-weaning mortality in the Px group was still significantly lower (5.2%) than the control group (P<0.001). This is a 22% reduction in PWM during a trial that provided extra staff to monitor the pigs. In a commercial farm with normal staffing levels, the reduction in PWM is likely to be more pronounced.

### Effect of Px in Gruel at Weaning

### Px Gruel at Weaning (TON-UK-032)

Piglets making the transition from weaning may find it difficult to adjust to dry food.

The aim of this study was to determine the effect of feeding gruel to pigs in the peri-weaning period. This study compared gruel made with Px to gruel made with water.

### Materials and Methods

In a farm located in Northern Ireland, 12 sows and their litters were enrolled in the study. Starting at approximately 24 hours after birth (Day 2 of life), Px litters were given 500 mL of Px in an open pan, in their farrowing crate. Control litters were given no extra supplementation. All litters had access to fresh water through a drinker nipple. Px litters continued to receive Px up through Day 8 of life. On Day 25 and 26, Px litters were again given 500 ml Px solution in an open pan. On Day 27, Px litters were given a gruel consisting of Px mixed with creep feed in the ratio of 1.5 L Px to 1 kg dry feed. Control litters received a similar gruel but made with plain water. On Day 28, all piglets were weaned and moved into weaning pens. Px piglets continued to receive Px-gruel for the next 3 days until Day 32, while Control pigs were given gruel made with water for the same period. Dry feed was available to both groups ad lib. Piglet ID, Sow ID, treatment group and gender were recorded for each pig. Pigs were individually weighed on Day 2, Day 8, Day 25, Day 32, Day 63 (7 weeks) and day 94 (13 weeks). Weight gain and average daily gain was calculated for each time interval and analysed with R software. Averages were calculated as least squares means (LSM). 95% confidence intervals were calculated from these. P values <0.05 were considered statistically significant.

### Results

The mean (LSM) weight gain per pig in the peri-weaning period (day 25-32) was 1.09±0.073 kg in the control group (n=63) and 1.46±0.068 kg in the Px group (n=49). This difference was highly significant (P<0.001). No clear differences were seen at day 63, but by day 94 (13 weeks of age) the Px pigs were an average of 4.78 kg heavier than controls (P=0.0002).

TABLE 4
Weight Gain through Day 94

| | | | Difference | |
|---|---|---|---|---|
| | Px-gruel | Water-gruel | (kg) [lb] | P value |
| BW at 25 days of age | 7.47 ± 0.245 | 7.27 ± 0.263 | | 0.589 |
| BW at 32 days of age | 8.93 ± 0.259 | 8.36 ± 0.279 | | 0.136 |
| Gain d 25 − 32 | 1.46 ± 0.068 | 1.09 ± 0.073 | 0.370 [0.8] | <0.001 |
| BW at 63 days of age | 23.32 ± 1.983 | 21.4 ± 1.087 | | 0.398 |
| Gain d 25 − 63 | 15.85 ± 1.923, | 14.12 ± 0.9748, | | 0.4256 |
| | n = 73 | n = 63 | | |
| BW at 94 days of age | 46.77 ± 0.8465, | 41.99 ± 0.94, | 4.78 [10.5] | 0.0002 |
| | n = 71 | n = 62 | | |
| Gain d 25 − 94 | 39.19 ± 0.7476, | 34.7 ± 0.7619, | 4.49 [9.9] | <0.0001 |
| | n = 71 | n = 62 | | |

All weight values are reported as mean ± SE.

### Conclusions and Relevance

Pigs that were weaned with Px-gruel gained an extra 370 grams during the first week of weaning compared to those that were given water-gruel and tended to be heavier at day 32. Differences seen at day 63 (7 weeks) were then markedly significant by 94 days (13 wks) of age when the Px pigs weighed 4.78 kg more than the control pigs. This confirms the theory that using Px to support young pigs gives real economic returns.

### Px-Gruel Vs Water-Gruel Vs Dry Creep—Productivity

Piglets making the transition from weaning may find it difficult to adjust to dry food. The aim of this study was to determine the effect of feeding gruel to pigs in the peri-weaning period. This study compared gruel made with Px vs. gruel made with water vs. dry creep feed.

### Materials and Methods

In a farm located in Spain, 52 sows and their litters (608 piglets) were enrolled in the study. Starting at approximately 24 hours after birth (Day 2 of age), Px litters were given 500 mL of Px in an open pan, in their farrowing crate. Px litters continued to receive Px up through day 8 of age. Control litters were given no extra supplementation. All litters had access to fresh water through a drinker nipple. At 15 days of age, the litters were split into 3 subsets and given either dry creep feed, water-gruel or Px-gruel for the next 3 days (all feeds ad lib). All gruel was made using the ratio of 1.5 L of liquid (water or 3% Px solution) to 1 kg of dry feed. On day 19, pigs were weaned and sorted into pens by bodyweight (heavy, medium, light) while still remaining within their feed groups. Heavy was defined as >5.8 kg, medium 4.1-5.8 kg and light <4.1 kg. All pigs continued to receive their gruel or dry feed ad lib for at least 2 days after weaning and were then tapered off gruel over another 3-4 days according to body weight. Light and medium pigs were tapered more slowly. Feed intake was measured for each group and normalised to dry matter intake (DMI), which equalised differences between the volume and weight of the gruel and dry feed. Feed intake was calculated for the pre-weaning period, 7 days post-weaning and 14 days post-weaning. Average daily gain was calculated for the same periods. The number of pigs with positive ADG in the first week post-weaning was calculated. Averages were calculated as least squares means (LSM)±standard error (SE). P values <0.05 were considered statistically significant, with P>0.05 and <0.10 considered to be a trend. The number of pigs requiring antibiotic treatment during the study period was also recorded.

### Results

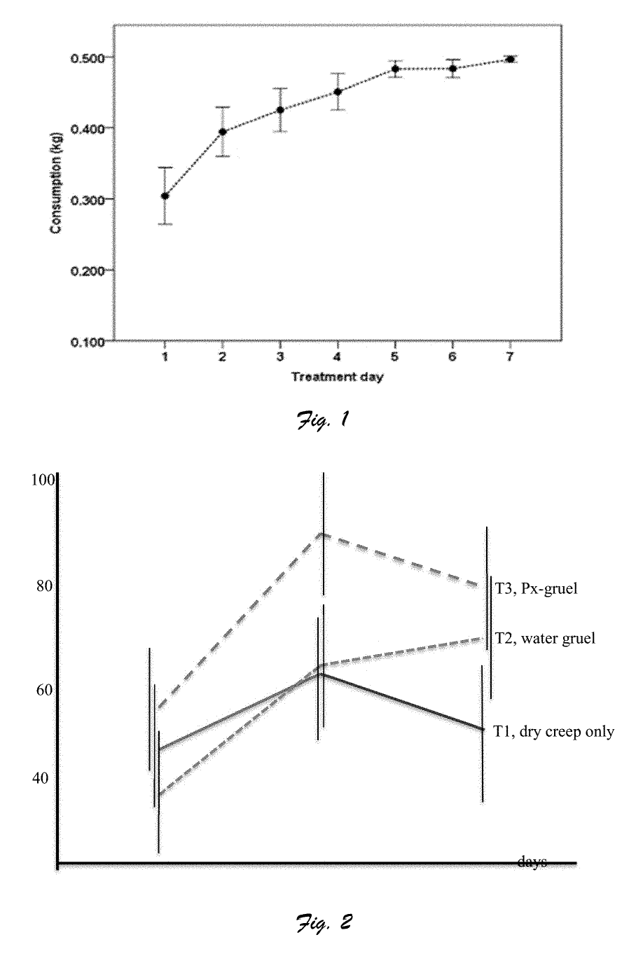

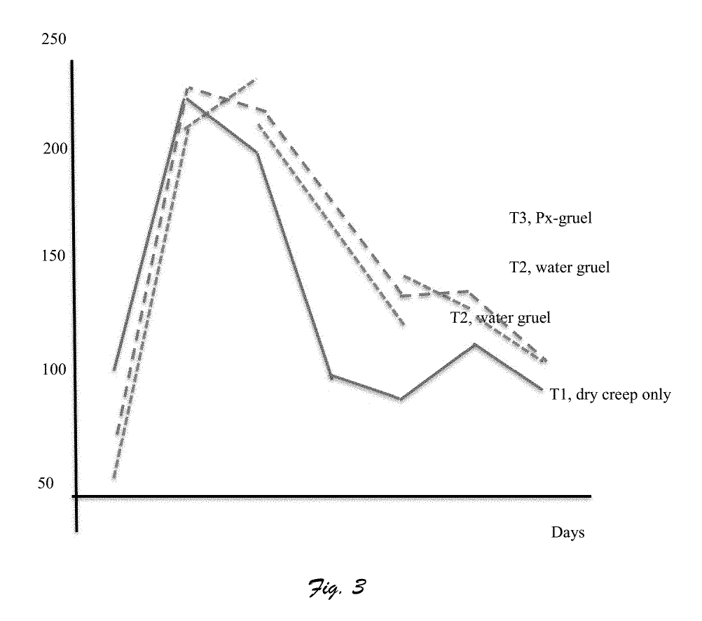

In the pre-weaning period, pigs who received Px-gruel had the greatest DMI as shown in FIG. 2 which presents Daily DMI from 1 to 3 Days before Weaning (g/pig/day).

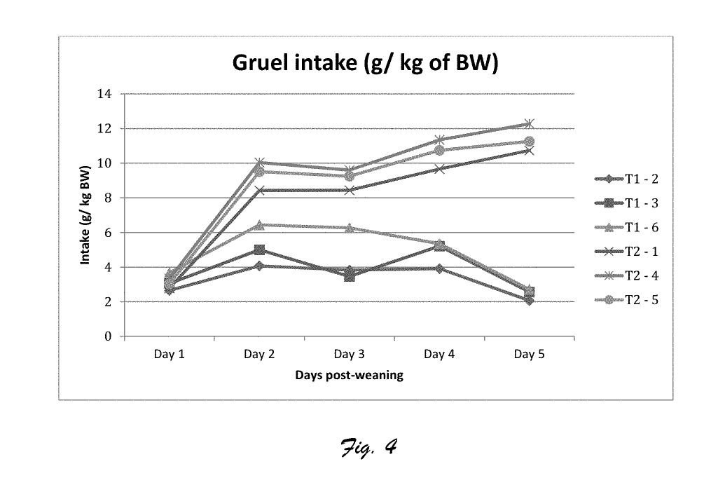

The Px-gruel group had an average DMI of 54±3.6 g/kg BW in the pre-weaning period, which was significantly higher than the water-gruel group (40±3.5 g/kg BW) or the dry creep group (39±4 g/kg BW) (P<0.05). This pattern of consumption continued over the next 6 days post-weaning as shown below. Reference is made to FIG. 3 presenting Daily DMI intake for 6 days Post-Weaning (g/pig/day).

The DMI was also tracked for the pens of Heavy, Medium and Light pigs. The table below shows total DMI of gruel and creep feed/kg BW in the first week and second weeks after weaning.

The most marked differences were seen in the Light and Medium pigs in the first week after weaning. Both gruel groups had significantly higher DMI compared to the dry feed group.

TABLE 5
Average daily DMI of gruel and/or creep feed per kg BW (g/kg
BW), by group and size

| | | | | | Water- | | | |
|---|---|---|---|---|---|---|---|---|
| | Days | Dry creep (T1) | | | gruel (T2) | | Px-gruel (T3) | |
| | Post- | LSM | | CI | LSM | CI | LSM | CI |
| Size | Weaning | (SE) | | 95% | (SE) | 95% | (SE) | 95% |
| H | 0 to 6 | 220 | | 194- | 233 | 207- | 222 | 197- |
| | | (13.2) | | 245 | (13.6) | 260 | (12.4) | 246 |
| H | 7 to 14 | 236 | | 180- | 225 | 168- | 249 | 197- |
| | | (28.3) | | 291 | (29.1) | 282 | (26.5) | 301 |
| M | 0 to 6 | 171^{b} | | 156- | 211^{a} | 198- | 198^{ab} | 186- |
| | | (7.5) | | 185 | (6.6) | 224 | (6.3) | 210 |
| M | 7 to 14 | 214 | | 183- | 233 | 205- | 228 | 202- |
| | | (16.1) | | 246 | (14.1) | 261 | (13.4) | 254 |
| L | 0 to 6 | 180^{b} | ^{t} | 153- | 224^{ab} | 198- | 254^{a} | 231- |
| | | (13.9) | | 207 | (13.2) | 250 | (11.7) | 277 |
| L | 7 to 14 | 198 | | 140- | 217 | 162- | 201 | 152- |
| | | (29.7) | | 256 | (28.1) | 272 | (25.0) | 250 |

The differences in DMI were then reflected in ADG. The Px-gruel group as a whole had a significantly larger number of pigs who gained weight in the first week after weaning.

TABLE 6
Number and percentage of piglets with positive ADG
in the first week post-weaning, by treatment group

| | Dry creep (T1) | | Water-gruel (T2) | | Px-gruel (T3) |
|---|---|---|---|---|---|
| ADG ≤ 0 | 31 | (22%) | 41 | (23%) | 28 (14%) |
| ADG > 0 | 113^{ab t} | (78%) | 139^{b} | (77%) | [symbol] |

^{a,b}different superscript in the same row indicates statistical differences (P ≤ 0.05).
^{t} in the same row indicates statistical tendency (P ≤ 0.1)

When broken down by size, the Px-gruel Medium and Light piglets also out-gained the other treatment groups. 88% of the Medium pigs and 56% of the Light pigs given Px-gruel had positive ADG in the first week post-weaning (P<0.05)

TABLE 7
Number and percentage of piglets with positive ADG in the first
week post-weaning, by treatment group and size (H, M and L).

| Size | | Dry creep (T1) | | Water-gruel (T2) | | Px-gruel (T3) | |
|---|---|---|---|---|---|---|---|
| H | ADG ≤ 0 | 9 | (24%) | 15 | (31%) | 11 | (30%) |
| | ADG > 0 | 29 | (76%) | 34 | (69%) | 26 | (70%) |
| M | ADG ≤ 0 | 17 | (25%) | 17 | (19%) | 12 | (12%) |
| | ADG > 0 | 51^{b} | (75%) | 74^{ab} | (81%) | 88^{a} | (88%) |
| L | ADG ≤ 0 | 5 | (13%) | 9 | (23%) | 5 | (8%) |
| | ADG > 0 | 33^{ab} | (87%) | 31^{b} | (78%) | 56^{a} | (92%) |

^{a,b}different superscript in the same row indicates statistical differences (P ≤ 0.05).

Odds ratios were also calculated for each group to assess the likelihood of an effect. Light pigs in the Px-gruel group were 3.25 (225%) times more likely to have positive ADG than the dry creep group (P=0.050) and 1.7 times (70%) more likely to have positive ADG than the water-gruel group (P=0.43). Medium pigs in the Px-gruel group were 2.44 (144%) times more likely to have positive ADG than the dry creep group (P=0.032) and 1.68 (68%) times more likely to have positive ADG than the water-gruel group (P=0.202).

Interestingly, dry-creep pigs were 1.7 (70%) times more likely to be treated with antibiotics in the post-weaning period (P=0.068).

### Conclusions and Relevance

Feeding Px-gruel in the peri-weaning period had a significant impact on the feed intake of all weight classes. The ADG of medium-weight and light-weight pigs was significantly improved. Px helped those pigs to compensate. The reduction in antibiotic requirements suggests another benefit of Px during weaning and will be a subject of further investigations for Tonisity.

### Effect of Px Gruel on Feed Intake and ADG Post-Weaning

Px is a highly palatable liquid that can be mixed with dry feed. Pigs at weaning often have reduced feed intake in the first few days after weaning. The aim of this pilot study was to see if a gruel made with Px and creep feed would result in increased feed intake and weight gain in the immediate post-weaning period.

### Materials and Methods

At weaning, 150 pigs were individually weighed and then randomly allocated to 6 pens, each containing 25 pigs. 3 pens received Px-gruel and 3 pens received water-gruel for 5 days after weaning. The gruel was mixed with the farm's usual creep feed, using 1.5 L of either 3% Px solution or water to 1 kg of feed and poured into extra creep feeders. Dry creep feed was also available in each pen. The quantity of gruel and dry creep feed consumed was weighed and calculated daily. On day 5 post-weaning, pigs were again individually weighed and ADG was calculated. The percentage of pigs achieving a positive ADG was also calculated. Gruel and dry feed consumption per pen was used to calculate gruel and feed intake per pig and per kg body weight (BW).

### Results

Fifty of the 75 pigs (66%) in the Px-gruel group had a positive ADG over the 5 days, compared to 18 of 75 pigs (25%) in the Water-gruel group. The ADG in the Px-gruel pens is shown in the table below.

TABLE 8
Average Daily Gain (g/day)*

| | Px-Gruel | Water-Gruel |
|---|---|---|
| Pen 1 | 6 ± 77 | −21 ± 108 |
| Pen 2 | −29 ± 133 | −85 ± 109 |
| Pen 3 | −163 ± 419 | −137 ± 84 |

*Values are mean ± standard deviation

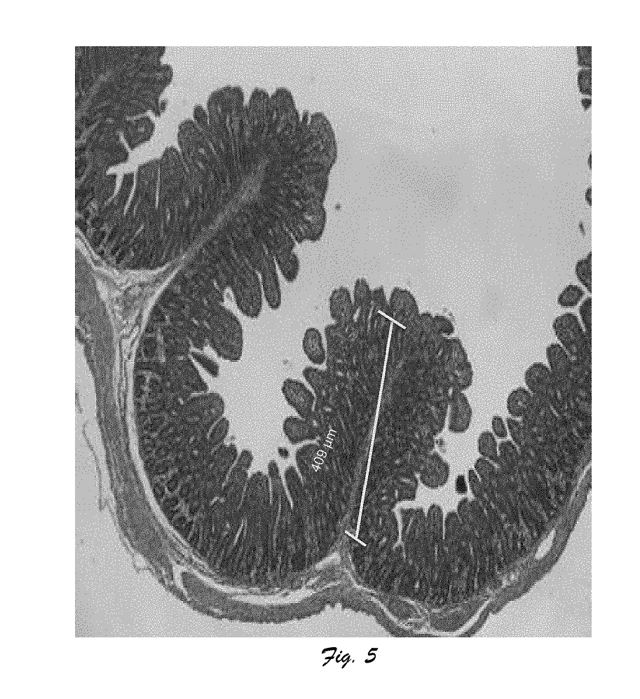

The Px-gruel pens consumed their gruel at an average of 362 g/pig (43 g/kg BW) compared to the Water-gruel pens that consumed an average of 158 g/pig (20 g/kg BW). Dry feed consumption in the Px-gruel pens averaged 9 g/pig compared to 20.6 g/pig in the Water-gruel pens. The intake in the Px-gruel pens is shown in the table below and in FIG. 4 showing Gruel Intake Comparison.

TABLE 9
Gruel and Feed Intake (g/pig)

| Px-Gruel | | Water-Gruel | |
|---|---|---|---|
| Gruel intake, g/pig | Feed Intake g/pig | Pen | Intake g/pig |
| 369 | 9 | 155 | 12 |
| 360 | 7 | 157 | 20 |
| 357 | 11 | 164 | 30 |

### Conclusions

Gruel made with creep feed and a 3% solution of Px is highly palatable. The consumption of gruel made with Px was twice that of water-gruel. Using Px-gruel resulted in increased average daily gain and a higher percentage of pigs that achieved positive ADG in the 5-day post-weaning period. A corresponding decrease in dry feed consumption was seen, but was offset by the increased consumption of gruel.

### Intestinal Morphology

Px from Days 2-8 of Age—Effect Upon Intestinal Morphology and PWM

The aim of this study was to determine whether or not giving Px to pigs in the first week of life had an impact on pre-weaning mortality and intestinal morphology. The usual approach to assessment of intestinal structure begins with microscopic measurements of the intestinal villi, which are the finger-like projections that line the small intestine and serve as the anchor for all intestinal cells. Villus height is used as a marker of intestinal health, and villus height decreases in the presence of inflammation or bacteria. The thickness of the mucus layer is also measured as an indicator of intestinal health and its ability to resist infection. The mucus layer helps to prevent bacterial adhesion and also contains antimicrobial molecules that are secreted by the intestinal cells.

### Materials and Methods

This study was approved by the ethics committee of the University of Lleida, Spain. In a farm located in Spain, 12 sows and their litters (134 piglets) were enrolled in the study. Starting at approximately 24 hours after birth (Day 2 of life), Px litters were given 500 mL of Px in an open pan, in their farrowing crate. Control litters were given no extra supplementation. All litters had access to fresh water through a drinker nipple. Px litters continued to receive Px 500 mL/litter up through Day 8 of life. On day 9 and day 21 of age, a total of 36 piglets (18 per group) were slaughtered for gut morphological assay. Selection of the piglets was done based on their ADG from initial weight to slaughtering time: day 2 to day 9 or day 2 to day 21. Within each litter, quartiles (25%, 50% and 75%) of the ADG were used to select three piglets per litter, from six litters (three per treatment) at day 9 and another three piglets per litter from the other six litters (three per treatment) at day 21. The piglets closest to each quartile were selected⁹ Piglets were slaughtered by intracardiac injection of T-61. The abdominal cavity was opened and gut segments (6-8 cm in length) for microscopy were obtained at proportional distances ˜10, ˜50 and ˜90% along the whole length of the small intestine, from the gastric pylorus to the ileo-caecal valve (duodenal, jejunal and ileal sections). At each side, both ends of the tissue were opened lengthwise (1-2 cm) to allow a full contact of the formalin solution with the mucosa. Samples were fixed by immersion in 10% formalin solution. Transverse tissue samples were cut from each segment using a stereo microscope. Samples were transferred to a slide and stained with hematoxylin and eosin. Measurements were only taken from sections where the plane of section ran vertically from the tip of a villus to the base of an adjacent crypt. Ten of the tallest well-orientated villi (complete distance from muscle layer to tip), 10 associated crypts (taken as the distance between the villus base and the muscular layer), the villus width (one third down from the tip of the villus) and the crypt width (in the middle of the crypt) were measured. Villus height (μm), crypt depth (μm), and intestinal mucus thickness (μm) were measured. Villus height/crypt depth ratio was calculated. Villus density (number/mm) and crypt density (number/mm) were measured. The average measurements per slide were used as the experimental observation as published by Berkeveld et al. see Berkeveld M, Langendijk P, Soede N M et al. Improving adaptation to weaning: effect of intermittent suckling regimens on piglet feed intake, growth, and gut characteristics. J Anim Sci 2009; 87:3156-66 which is incorporated herein as a reference.

### Statistical Analysis

Gut morphology variables were analysed with a generalised linear model using the package GLM for R software. Treatment group, intestinal section and age were considered as fixed effects. Interactions were explored but were not significant, so were not included in the model. Pre-weaning mortality was compared by logistic regression using the package GLM for R software, with the treatment group as the fixed effect.

### Results Pre-Weaning Mortality

Pigs that received Px in the first week of life had significantly lower pre-weaning mortality (4% vs 19%, P=0.015). This was a 75% reduction in PWM.

Intestinal Morphology

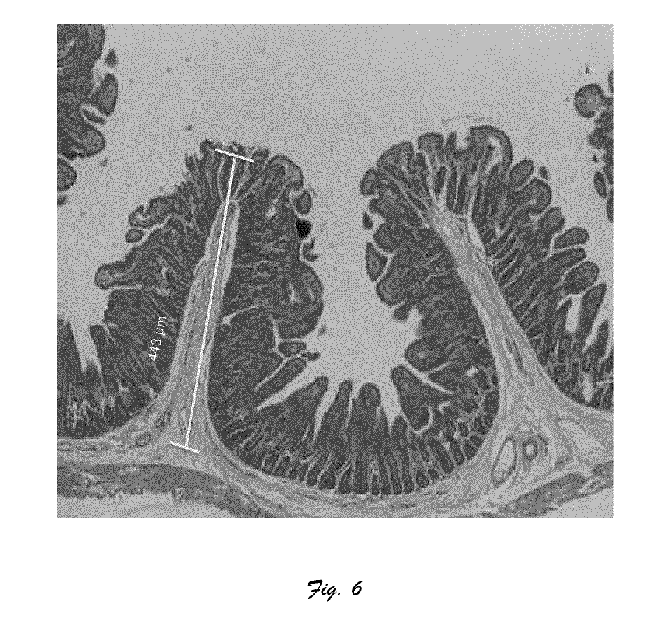

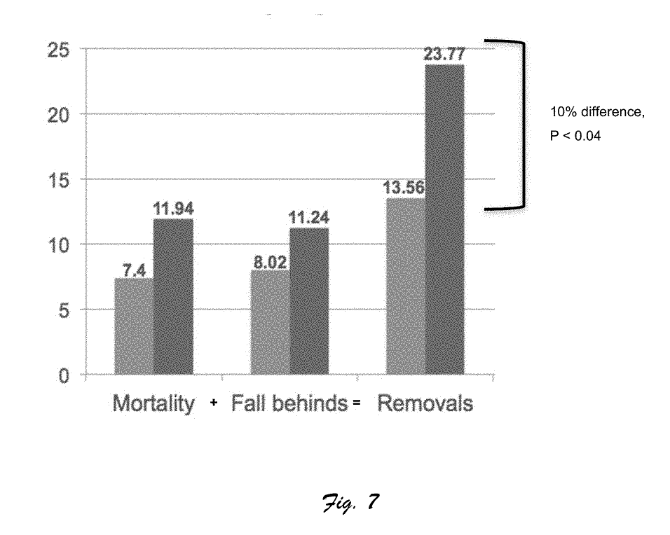

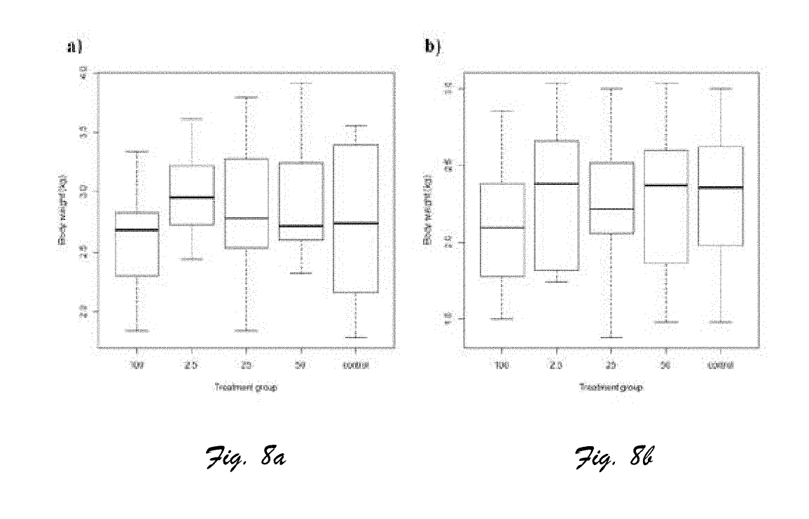

Villus height, villus height/crypt depth ratio and intestinal mucus thickness were significantly higher (P<0.05) in pigs that received Px in the first week of life, as shown in FIG. 4: Villi from Control Group (Jejunum); and FIG. 5: Villi from Px Group (Jejunum).

TABLE 10
Intestinal Morphology Pre-Weaning

| | | | Villus | Intestinal | Villus | Crypt |
|---|---|---|---|---|---|---|
| | Villus | Crypt | height/ | mucus | density | density |
| | height | depth | crypt depth | thickness | (No./ | (No./ |
| | (μm) | (μm) | ratio | (μm) | mm) | mm) |
| Control | 409 | 139 | 3.6 | 536 | 9.3 | 20.6 |
| Px | 443 | 148 | 3.6 | 584 | 8.7 | 18.3 |
| P-value | <0.001 | 0.199 | 0.962 | 0.087 | 0.037 | 0.003 |

### Conclusions and Relevance

Pigs that received Px during the first week of life had significantly taller villi than pigs that did not and tended to have a thicker mucus layer. Pigs that received Px during the first week of life had significantly lower pre-weaning mortality compared to those that did not.

### Px-Gruel Vs Water-Gruel Vs Dry Creep—Intestinal Morphology

This study was part of the previous study (16-003-1) that compared gruel made with Px vs. gruel made with water vs. dry creep feed. A subset of pigs was used to assess the effect of Px supplementation upon intestinal morphology.

### Materials and Methods

This study was approved by the ethics committee of the University of Lleida, Spain. In a farm located in Spain, 52 sows and their litters (608 piglets) were enrolled in the study. Starting at approximately 24 hours after birth (Day 2 of life), Px litters were given 500 mL of Px in an open pan, in their farrowing crate. Control litters were given no extra supplementation. All litters had access to fresh water through a drinker nipple. Px litters continued to receive Px up through Day 8 of life. Starting on day 15 of life, the litters were split into 3 subsets and given either dry creep feed, water-gruel or Px-gruel for the next 3 days (all feeds ad lib). All gruel was made using the ratio of 1.5 L of liquid (water or 3% Px solution) to 1 kg of dry feed. On day 19, pigs were weaned and sorted into pens by bodyweight (heavy, medium, light) while still remaining within their feed groups. Heavy was defined as >5.8 kg, medium 4.1-5.8 kg and light <4.1 kg. All pigs continued to receive their gruel or dry feed ad lib for at least 2 days after weaning and were then tapered off gruel over another 3-4 days according to body weight. Light and medium pigs were tapered more slowly. A total of 36 piglets (12 per group) were slaughtered after weaning for gut morphological assay. Eighteen (18) piglets (six per group) were selected on day 24 and on day 28 based on their BW at day 18. Within the initial group (Px or no Px in week 1) and treatment group (Px-gruel, water-gruel or dry creep feed), quartiles (25%, 50% and 75%) of the BW at weaning were used to select six piglets per group of treatment at 5 and 9 days after weaning, corresponding to days 24 and 28 of age. The piglets closest to each quartile were selected. Piglets were slaughtered by intracardiac injection of T-61. The abdominal cavity was opened and gut segments (6-8 cm in length) for microscopy were obtained at proportional distances ˜10, ˜50 and ˜90% along the whole length of the small intestine, from the gastric pylorus to the ileo-caecal valve. These distances resulted in duodenal, jejunal and ileal sections. At each site, both ends of the tissue were opened lengthwise (1-2 cm) to allow a full contact of the formalin solution with the mucosa. Samples were fixed by immersion in 10% formalin solution. Transverse tissue samples were cut from each segment using a stereo microscope. Samples were transferred to a slide and stained with hematoxylin and eosin. Measurements were only taken from sections where the plane of section ran vertically from the tip of a villus to the base of an adjacent crypt. Ten of the tallest well-orientated villi (complete distance from muscle layer to tip), 10 associated crypts (taken as the distance between the villus base and the muscular layer), the villus width (one third down from the tip of the villus) and the crypt width (in the middle of the crypt) were measured. Villus height (μm), crypt depth (μm), and intestinal mucus thickness (μm) were measured. Villus height/crypt depth ratio was calculated. Villus density (number/mm) and crypt density (number/mm) were measured. The average measurements per slide were used as the experimental observation as published by Berkeveld et al. cited above.

### Statistical Analysis

Gut morphology variables were analysed with a generalised linear model using the package GLM for R software. Interactions between weight classes, litter Px treatment, weaning feed treatment, study day and intestinal section were explored but were not significant, so were not included in the model.

### Results

Villus height, villus height/crypt depth ratio and intestinal mucous thickness were significantly higher (P<0.05) in pigs that received Px in the first week of life.

TABLE 11
Comparison of Intestinal Morphology Parameters Post-Weaning

| | | | Villus | | | |
|---|---|---|---|---|---|---|
| | | | height/ | Intestinal | Villus | Crypt |
| | Villus | Crypt | crypt | mucus | density | density |
| | height | depth | depth | thickness | (No./ | (No./ |
| | (μm) | (μm) | ratio | (μm) | mm) | mm) |
| Control | 249 ± | 220 ± | 1.2 ± | 435 ± | 8.0 ± | 20.5 ± |
| | 9.3 | 6.4 | 0.05 | 14.2 | 0.15 | 0.71 |
| Px | 291 ± | 226 ± | 1.4 ± | 481 ± | 7.6 ± | 21.8 ± |
| | 10.4 | 7.1 | 0.06 | 15.9 | 0.17 | 0.79 |
| P-value | 0.003 | 0.512 | 0.018 | 0.033 | 0.064 | 0.211 |

### Conclusions and Relevance

Regardless of the feed given at weaning, pigs that received Px during the first week of life had significantly better intestinal morphology in the post-weaning period. The development of the villi seen when Px is given from day 2-8 of age continued through to the post-weaning stage, showing the efficiency and impact of Tonisity Px formula on intestinal development.

### Scour

Px in Piglets with Scour

Piglets suffering from diarrhoea may become dehydrated. Px is an isotonic solution containing balanced electrolytes and protein, and may be used for rehydration and support in such situations. The aim of this study was to determine the effect of Px on suckling pigs with scours.

### Materials and Methods

In a farm located in North Carolina, USA, with documented Clostridium perfringens, E. coli and rotavirus, 79 litters (898 pigs) were prospectively enrolled in the study when they developed scour between 2-4 days of age. Once enrolled, piglets were individually ear-tagged. Odd-numbered piglets within a litter were given 2 mL of 3% Px solution by mouth twice daily×5 days. Even-numbered piglets received no oral supplements. All piglets received standard farm treatment (1 mg/kg ceftiofur IM×3 days) to control secondary pathogens. Fecal scores were recorded daily for individual pigs, where 0=normal feces, 1=pasty feces and 2=liquid feces. Total fecal scores were summed for each piglet. Pigs were defined as ‘recovered’ if their fecal score was 0 on day 5. All pigs were individually weighed when enrolled, at 8 days after enrolment and at 18 days after enrolment. Any pigs that died or were removed to a nurse sow were recorded. Morbidity, mortality, sick animals and recovered animals were compared by logistic regression using a generalised linear model of R software, with piglet as the experimental unit and treatment as a fixed effect. Body weight and average daily gain were analysed as a general linear model with treatment, gender, room and date of inclusion as fixed effects. Fecal scores from inclusion to study day 5 were analysed using the Wilcoxon test. Results were considered significant at P≤0.05 and considered a trend at P>0.05 and P≤0.10. Odds ratios were calculated.

### Results

The percentage of pigs that had recovered after 5 days was higher in the Px group (71%) compared to the control group (62%). Odds ratio calculation indicated that pigs receiving Px were 1.48 (48%) times more likely to recover in that period of time (P=0.112). The percentage of pigs that were culled as fall-behinds was higher in the control group (8%) compared to the Px group (5%). Odds ratio calculation indicated that control pigs were 1.59 times (59%) more likely to be culled (P=0.098).

### Conclusions and Clinical Relevance

This was the second study in a proof-of-concept series designed to investigate the role of Px in supporting piglets through episodes of neonatal scour. It is interesting that even 4 mL daily had an effect. Further studies are planned to investigate the optimum volume and timing of delivery for those farms in which twice-daily dosing is too labor-intensive.

Px in Piglets with Scour (T20)

Piglets suffering from diarrhoea may become dehydrated. Px is an isotonic solution containing balanced electrolytes and protein, and may be used for rehydration and support in such situations. The aim of this study was to determine the effect of Px on suckling pigs with scours.

### Materials and Methods

In a farm located in the midwestern USA, with historically-documented E. coli and rotavirus, 20 litters (268 pigs) were prospectively enrolled in the study if they developed scour between 2-4 days of age. Once enrolled, piglets were individually ear-tagged. Odd-numbered piglets within a litter were given 2 mL of Px by mouth twice daily. Even-numbered piglets were given 2 mL of water by mouth twice daily. All piglets received standard farm treatment to control secondary pathogens. All pigs were individually weighed when enrolled, and again at day 18. Any pigs that died or were removed to a nurse sow were recorded. Data were analyzed as a randomized complete block design using the PROC MIXED procedure of SAS with piglet as the experimental unit and treatment as a fixed effect. Results were considered significant at P≤0.05 and considered a trend at P>0.05 and P≤0.10.

### Results

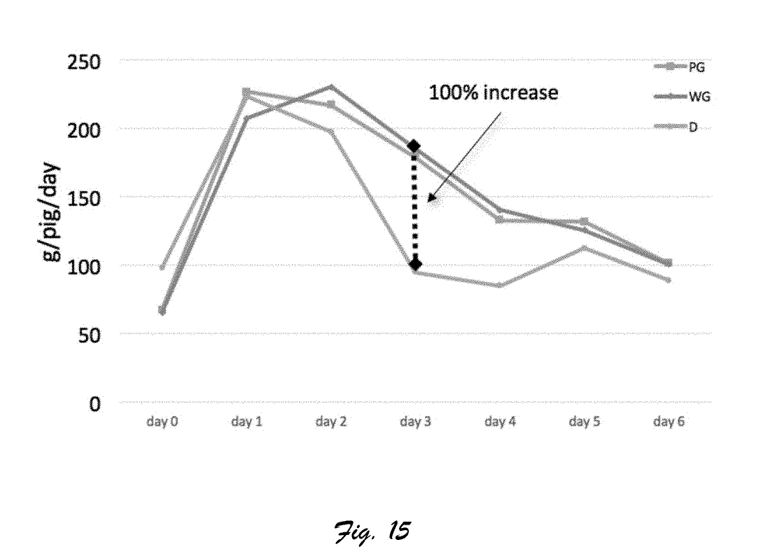

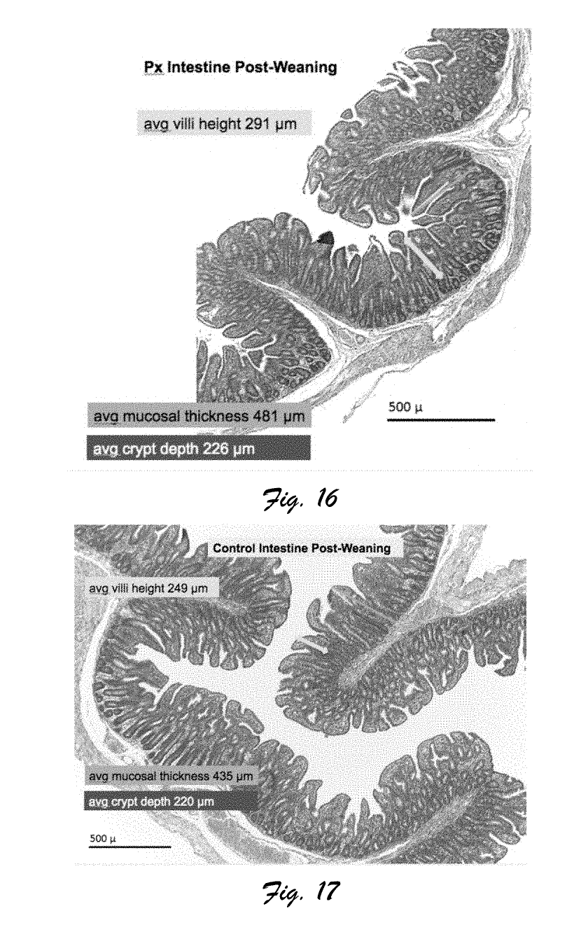

Pre-weaning mortality for the Px group was 7.40%, compared to the control group which had a mortality of 11.94% (P=0.21). Similarly, the percentage of pigs that were removed as fall-behinds was lower in the Px group (8.02%) compared to the control group (11.24%) (P=0.38). When mortality and fall-behinds are combined, the total percentage was significantly lower in the Px group (13.56%) compared to the water group (23.7%) (P=0.04). Reference is now made to FIGS. 6, 16 and 17, that compare Px v Control in Scouring Piglets.

### Conclusions

These results show that even small amounts of Px are helpful in the support of piglets with scour, and can have a significant impact upon removals.

### Palatability Aid

### Palatability of Water-Soluble Antibiotic in Nursery Pigs

Px is a novel isotonic solution that provides both rehydration and protein. Pilot studies have shown that it is highly palatable to both suckling and weaned pigs. Pigs are often given oral medications such as antibiotics in water, but many of those medications are poorly accepted. The aim of this study by Tonisity was to assess whether or not Px could be used to increase the palatability of medication in weaned pigs.

### Materials and Methods

One hundred and ninety eight healthy piglets of approximately 21 days of age were housed in six pens at weaning. Piglets were grouped by size in each pen. Each pen had an automatic drinker. Each pen was also provided with a bowl drinker that was connected to a 20 L carbuoy. The carbuoys were filled each day with a solution of antibiotic in water or antibiotic in a 3% solution of Px. The antibiotic used was Neomycin (sulfate) 100 mg; colistin (sulfate) 40 mg, commercially available by Maymó Lab. As a trademarked product named Coliphur™. Five pigs were randomly chosen from each pen to establish an average body weight. This average body weight was then used to calculate the dose of Coliphur™ required (0.1 mL/kg BW/day) and the volume of water required (10% of body weight/day) for each pen. Starting at 3 days after weaning, pens were allocated to receive either antibiotic in water or antibiotic in Px for 2 days. After 2 days, pens were allocated to the opposite treatment in a crossover design (Table 1).

TABLE 12
Allocation of Treatment Groups

| | No. | of TREATMENT DAY | | | |
|---|---|---|---|---|---|
| PEN | pigs | 1 | 2 | 3 | 4 |

| | | | |
|---|---|---|---|
| 1 | 19 | antibiotic + water | antibiotic + 3% Px solution |
| 2 | 40 | antibiotic + water | antibiotic + 3% Px solution |
| 3 | 37 | antibiotic + water | antibiotic + 3% Px solution |
| 4 | 38 | antibiotic + 3% Px solution | antibiotic + water |
| 5 | 40 | antibiotic + 3% Px solution | antibiotic + water |
| 6 | 24 | antibiotic + 3% Px solution | antibiotic + water |

All medicated solutions were made fresh each morning. The volume of unconsumed solution from the previous day was measured each morning.

### Results

Pigs receiving the antibiotic in Px consumed 94% of their calculated intake volume, but the pigs receiving the antibiotic in water consumed only 33% of their calculated intake volume. Pens in the Px+antibiotic group achieved either 0.09 or 0.10 mL/kg BW of medication on 11 of the 12 treatment days, but none of the pens in the water+antibiotic group received the recommended dose of antibiotic on any day.

TABLE 13
Consumption of Medicated Solutions in Pens

| | | | MEDICATED WATER (L) | | | | NUMBER | COLIPHU [symbol] |
|---|---|---|---|---|---|---|---|---|
| | | | ADDED | | INTAKE | | OF | DOSE |
| PE [symbol] | TREATM [symbol] | DA [symbol] | [symbol] | LEFTOV [symbol] | [symbol] | | [symbol] | [symbol] |
| 1 | control | 1 | 9.79 | 6.09 | 3.70 | 38% | 19 | 0.04 |
| 1 | control | 2 | 9.79 | 6.46 | 3.33 | 34% | 19 | 0.04 |
| 1 | Px | 3 | 9.83 | 0.00 | 9.83 | 100% | 19 | 0.10 |
| 2 | control | 1 | 25.22 | 17.20 | 8.02 | 32% | 40 | 0.03 |
| 2 | control | 2 | 25.22 | 17.85 | 7.37 | 29% | 40 | 0.03 |
| 2 | Px | 3 | 25.38 | 3.00 | 22.38 | 88% | 40 | 0.09 |
| 3 | control | 1 | 23.81 | 22.99 | 0.82 | 3% | 37 | 0.00 |
| 3 | control | 2 | 23.81 | 9.74 | 14.07 | 59% | 37 | 0.06 |
| 3 | Px | 3 | 23.64 | 1.44 | 22.19 | 94% | 37 | 0.09 |
| 4 | Px | 1 | 18.01 | 6.42 | 11.59 | 64% | 38 | 0.07 |
| 4 | Px | 2 | 17.76 | 0.39 | 17.37 | 98% | 37 | 0.10 |
| 4 | control | 3 | 17.76 | 13.25 | 4.51 | 25% | 36 | 0.03 |
| 5 | Px | 1 | 24.30 | 2.19 | 22.11 | 91% | 40 | 0.09 |
| 5 | Px | 2 | 24.30 | 0.40 | 23.90 | 98% | 40 | 0.10 |
| 5 | control | 3 | 24.30 | 9.14 | 15.17 | 62% | 40 | 0.06 |
| 6 | Px | 1 | 8.00 | 0.18 | 7.82 | 98% | 24 | 0.10 |
| 6 | Px | 2 | 8.00 | 0.05 | 7.95 | 99% | 24 | 0.10 |
| 6 | control | 3 | 10.52 | 8.41 | 2.11 | 20% | 23 | 0.02 |

[symbol]

indicates data missing or illegible when filed

### Conclusions

Px was effective at increasing the amount of medication consumed to the recommended dosing level of 0.1 mL/kg. While it is important that antibiotics are prescribed only when necessary, it is also important that the required dose be delivered. Further palatability tests using Px with other medications are warranted.

### Palatability of Water-Soluble Antibiotic in Suckling Pigs

Young piglets suffering from scour may benefit from rehydration. Some producers use oral antibiotics in the treatment of scour, but these antibiotics are sometimes unpalatable. The objective of this study was to determine whether Px would aid the consumption of medication in suckling piglets.

### Materials and Methods

Forty sows and their litters were randomly allocated to one of four treatment groups based on parity. The treatment groups were water (W), water+Coliphur™ (W+C), and Px+Coliphur™ (Px+C). The antibiotic used was Coliphur™, which is a mixture of neomycin and polymyxin B. All litters received 500 mL of their designated solution in an open pan, once daily, from day 2-7 of life. All piglets had access to an automatic drinker. The amount of solution consumed was recorded daily for each litter, and an average intake per piglet was calculated daily. On the day of farrowing, piglets were individually ear-tagged and weighed. Piglets were again weighed at day 7, and average daily gain was calculated. Mortality was recorded each day.

### Results

The control group receiving plain water (W) had the highest average intake of any group, consuming 180±16.8 mL/pig over the 5 days of treatment. The Px+C group consumed an average of 162±16.9 mL/pig, which was not significantly different from the control Water group. The Water+C group had the lowest intake of any group, averaging 102±17.7 mL/pig.

TABLE 14
Consumption of Medicated Solutions in Litters

| Group | Mean ± SE (mL/pig) |
|---|---|
| Water | 180 ± 16.8 |
| Px + Coliphur TM | 162 ± 16.9 |
| Water + Coliphur TM | 102 ± 17.7 |

### Conclusions

Px+Coliphur™ was significantly more palatable than Water+Coliphur™. The volume of Px+Coliphur™ consumed was not significantly different from plain water, suggesting that piglets find Px a very palatable product which may be used to deliver medications.

### Safety Data

### Dose Titration Trial

The purpose of this study was to assess piglets for any negative effect of Px when administered in the first week of life. The study evaluated the effect of Px on weight, scour incidence, gut bacteria populations, haematology and serum biochemistry, when it was administered to suckling piglets at different doses (2.5 mL, 25 mL, 50 mL and 100 mL) and for different durations (1 to 5 days) during first days of life.

### Materials and Methods

Ten sows and their litters (˜140 piglets) from two different farms located in central Spain were enrolled in the study.
Farm L was a typical farrow-to-nursery farm with 2500 Danbred sows, weekly farrowing and weaning at 21-26 days. Farm L has a history of scours during lactation with the following aetiology: bacterial aetiology (Clostridium difficile, Clostridium perfringens type C and Escherichia coli) and virus aetiology (Type A Rotavirus).
Farm A was a farrow-to-finish farm with 400 Landrace×Large White sows, with 4 weeks farrowing batches (80 sows per batch) and regular weaning at 21 days.

The farm has a low incidence of scours during lactation.

In order to minimize environmental effects all sows on each farm were allocated to the same farrowing room. Each litter/sow was assigned to one of the five treatment durations (from 1 to 5 days of treatment). Within each litter, piglets were randomly allocated to one of the five treatment groups (Control: 2 piglets; 2.5 mL: 3 piglets; 25 mL: 3 piglets; 3 piglets: 50 mL: 3 piglets; 100 mL: 3 piglets)

TABLE 15
Allocation of Piglets to Dose and Duration Groups

| Litter | Day −2 | Day −1 | Day 0 | Day 1 | Day 2 | Day 3 | Day 4 | Day 5 |
|---|---|---|---|---|---|---|---|---|
| Litter 1 | Farrowing | | (3) 2.5 ml | | | | | |
| | | | (3) 25 ml | | | | | |
| | | | (3) 50 ml | | | | | |
| | | | (3) 100 ml | | | | | |
| | | | (2) Control | | | | | |
| | | Blood¹ | | Blood² | | | | Weight₂ |
| | | Faeces¹ | | Faeces² | | | | |
| | | Weight₁ | | | | | | |
| Litter 2 | Farrowing | | (3) 2.5 ml | (3) 2.5 ml | | | | |
| | | | (3) 25 ml | (3) 25 ml | | | | |
| | | | (3) 50 ml | (3) 50 ml | | | | |
| | | | (3) 100 ml | (3) 100 ml | | | | |
| | | | (2) Control | (2) Control | | | | |
| | | Blood¹ | | | Blood² | | | Weight₂ |
| | | Faeces¹ | | | Faeces² | | | |
| | | Weight₁ | | | | | | |
| Litter 3 | Farrowing | | (3) 2.5 ml | (3) 2.5 ml | (3) 2.5 ml | | | |
| | | | (3) 25 ml | (3) 25 ml | (3) 25 ml | | | |
| | | | (3) 50 ml | (3) 50 ml | (3) 50 ml | | | |
| | | | (3) 100 ml | (3) 100 ml | (3) 100 ml | | | |
| | | | (2) Control | (2) Control | (2) Control | | | |
| | | Blood¹ | | | | Blood² | | Weight₂ |
| | | Faeces¹ | | | | Faeces² | | |
| | | Weight₁ | | | | | | |
| Litter 4 | Farrowing | | (3) 2.5 ml | (3) 2.5 ml | (3) 2.5 ml | (3) 2.5 ml | | |
| | | | (3) 25 ml | (3) 25 ml | (3) 25 ml | (3) 25 ml | | |
| | | | (3) 50 ml | (3) 50 ml | (3) 50 ml | (3) 50 ml | | |
| | | | (3) 100 ml | (3) 100 ml | (3) 100 ml | (3) 100 ml | | |
| | | | (2) Control | (2) Control | (2) Control | (2) Control | | |
| | | Blood¹ | | | | | Blood² | Weight₂ |
| | | Faeces¹ | | | | | Faeces² | |
| | | Weight₁ | | | | | | |
| Litter 5 | Farrowing | | (3) 2.5 ml | (3) 2.5 ml | (3) 2.5 ml | (3) 2.5 ml | (3) 2.5 ml | |
| | | | (3) 25 ml | (3) 25 ml | (3) 25 ml | (3) 25 ml | (3) 25 ml | |
| | | | (3) 50 ml | (3) 50 ml | (3) 50 ml | (3) 50 ml | (3) 50 ml | |
| | | | (3) 100 ml | (3) 100 ml | (3) 100 ml | (3) 100 ml | (3) 100 ml | |
| | | | (2) Control | (2) Control | (2) Control | (2) Control | (2) Control | |
| | | Blood¹ | | | | | | Blood² |
| | | Faeces¹ | | | | | | Faeces² |
| | | Weight₁ | | | | | | Weight₂ |

¹Samples collected before starting treatment.
²Samples collected the day after treatment ending.
(x) Number of piglets treated.
Weight₁: piglets' weight recorded on Day −1 (before treatment beginning).
Weight₂: piglets' weight recorded on Day 5.

Piglets were individually tagged, weighed and blood sampled on the day after farrowing (day 1). Pooled faecal samples were also collected on day 1. The incidence of scour and the severity of scour was recorded daily for each litter. Individual mortality was recorded daily. Treatment commenced on day 2 after farrowing. The allocated dose of Px was administered to each piglet once daily, orally, using a volumetric pump dispenser. Blood and faecal samples were taken from each pig on the day after their treatment ended. All blood samples were analysed for routine haematology and biochemistry (see table below) All piglets were individually weighed on day 7.

TABLE 16
Hematology and Biochemistry Parameters Measured

| Hematology Parameters | Biochemistry Parameters |
|---|---|
| Hematocrit, | Total protein, albumin, globulin |
| Total white cell count, | Urea, creatinine |
| % neutrophils, % monocytes, % | ALKP, ALT, AST, cholesterol |
| lymphocytes, % eosinophils | Amylase, lipase |
| | Calcium, Chloride, Phosphorus * |

* Sodium results not available due to sample handling issues

### Results

Body Weight and Average Daily Gain at day 7 of age—On both farms, pigs receiving 100 mL/day of Px tended to have decreased body weight and average daily gain. Other groups showed a large degree of variation and overlap in their body weights, with no detectable difference between treatment volume groups or duration of treatment. Reference is now made to FIGS. 8a and 8b: Piglets Body Weight (kg) on Day 6: (a)=Farm A, (b)=Farm L.

Mortality—only 4 pigs out of the 140 died. These 4 pigs were all from the same farm and same litter, and each pig was receiving a different dose of Px. Incidence and Severity of Scour—No piglets with scour were recorded at Farm A. At Farm L, 17 of the 70 pigs developed scour. There was no association between the incidence of scour and the treatment dose or duration.

Hematology and Biochemistry—There were significant differences between the pigs from Farm A compared to Farm L. However, within each farm, there were no clinically significant differences in the hematology or biochemistry parameters between the treatment volume or treatment duration.

### Conclusions

Px showed no deleterious health effects on piglets when given manually at doses that were between 3-5 times the usual intake volume. At high doses (100/pig/day×5 days), piglets showed decreased weight gain. This was attributed to competition for stomach capacity and milk intake, and would not be expected under normal conditions.

### Statistical Analysis

Unless otherwise noted, statistical analysis was performed using R software (R Core Team (2015). R: A language and environment for statistical computing. R Foundation for Statistical Computing, Vienna, Austria. ISBN 3-900051-07-0, currently available at URL http://www.R-project.org. Significance levels were set at P<0.05, with P>0.05 but less than 0.10 considered a tendency.

Example 6 discloses, inter alia, a composition as defined above, wherein the composition comprising, inter alia, all three ingredients as follows: (a) a sweet taste derived from glucose, the flavor enhancer steviol glycoside extract and from the amino acid glycine; (b) an acidic taste derived from citric acid monohydrate and tri-sodium citrate; and, (c) an umami taste, derived from the monosodium glutamate.

The composition of the present invention discloses, in a non-limiting manner, glycine as an essential amino acid for enterocytes, and also as a taste enhancer, see Yamamoto, Takashi. “Brain mechanisms of sweetness and palatability of sugars.” Nutrition reviews 61.s5, 2003, e.g., as cited in Page S5, para 5, which is incorporated herein as a reference.

The composition of the present invention discloses, in a non-limiting manner steviol glycosides. It encompasses the following characteristics: Safe to use; High intense sweeteners; Natural sweeteners; Low caloric; and, has no effect of osmolarity. It is in the scope of the invention wherein the term ‘sweetener’ refers to “those food additives, which are able to mimic the sweetness of sugar and which usually provide less energy. Some are natural extracts whilst others are synthetic. In the case of the latter they are also known as artificial sweeteners”, see García-Almeida, J. M., M. Gracia, and J. García Alemán “A current and global review of sweeteners; regulatory aspects.” Nutricion hospitalaria 28. Supl 4 (2013): 17-31; e.g., as cited from page 18 para 1, which is incorporated herein as a reference.

It is in the scope of the invention wherein steviol glycosides, stevia derivatives, the sweet components of the stevia leaf are utilized as natural non-caloric sweeteners which were approved by the EFSA and FDA; see García-Almeida, J. M., M. Gracia, and J. García Alemán. “A current and global review of sweeteners; regulatory aspects.” Nutricion hospitalaria 28. Supl 4 (2013): 17-31; e.g., as cited from page 19 para 2; and García-Almeida, J. M., M. Gracia, and J. García Alemán. “A current and global review of sweeteners; regulatory aspects.” Nutricion hospitalaria 28. Supl 4 (2013): 17-31; e.g., as cited from page 20 para 4; those two references are incorporated herein as references.

Stevia is derived form a plant source while sucralose is of a synthetic origin. Moreover, “Compounds from the stevia leaf are sweetening a variety of foods. Stevia's advantage over other natural mainstream sweeteners is that it contributes no calories to foods and beverages and has a zero glycemic load”, see “Sweet Options”, Food product design, vol. 21, no. 2, February 2011, e.g., as cited in page 6, para 2, which is incorporated herein as reference. Stevia, a sweetener of natural source, is preferable to synthetic sweeteners, which may exert health hazards, as cited: “For several years. High caloric sugars remain main source of sweetening agent. However, changing life-style and sugar-related health problems, such as obesity and dental caries, and un-suitability of sugars for diabetic patients, replacement of these high caloric sugars by low caloric intense sweeteners, have recently appeared in pharmaceutical and food industries, but their health hazards due to harmful side-effects restrict their utility, see Surana, S. J., et al. “Non-saccharide natural intense sweeteners—an overview of current status” Natural Product Radiance, pp. 270-278, (2006), cited in page 270, which is incorporated herein as reference.

While glucose and dextrose are caloric sweeteners, steviol is a non-caloric natural sweetener, see Table 1 as reproduced from García-Almeida, J. M., M. Gracia, and J. García Alemán “A current and global review of sweeteners; regulatory aspects.” Nutricion hospitalaria 28. Supl 4 (2013): 17-31; e.g., as cited from page 21 para 2, which is incorporated herein as reference. It is highly important to use a non-caloric sweetener as non-caloric sweeteners mimic the sweetness of sugar and, provide less energy. That is highly important for weight maintaining of the administered subjects, controlling blood sugar and carbohydrate levels, and also for maintaining the osmolarity (isotonicity) of the composition.

Typical usage levels of steviols are in the range of 0.02%-0.06%. Therefore, while dextrose provides high osmolarity, see “Sweet Options”, Food product design, vol. 21, no. 2, February 2011 which is incorporated herein as reference, steviol glycosides is useable as a sweet ingredient, without changing the osmolarity of the composition, thus keeping this composition isotonic. An isotonic solution is comparable to the osmolarity of body fluids and gastrointestinal cells and will contribute to faster absorption of the composition ingredients. Thus, it is highly important and advantageous to use an isotonic composition as compared with a non-isotonic composition. Isotonic drinks have been superior to water for example for soccer players which are dehydrated after exercise, see “isotonic CHO-electrolyte solutions have been shown to be superior to water in promoting fluid consumption” Hawley, John A., Steven C. Dennis, and Timothy D. Noakes. “Carbohydrate, Fluids and Electrolyte Requirements of the Soccer Player: A Review.” International journal of sport nutrition 4.3 (1994): 221-236, which is incorporated herein as reference.

Additionally, steviol glycosides are very sweet, and considered “high-intensity sweeteners”. There are also natural sweeteners. i.e., stevia, whose calories are insignificant compared to the quantities usually used for sweetening purposes. These are not carbohydrates, therefore they don't have a glycemic index, they are considered high-intensity sweeteners (HIS). Steviol glycosides are 200 to 400 times sweeter than sucrose. Steviol glycosides are sweeter than dextrose. Dextrose (also known as D-glucose) has mild sweetness, about 0.7 times the approximate sweetness of sugar, see Sweet Options”, Food product design, vol. 21, no. 2, February 2011, e.g., as cited in page 6, para 2, which is incorporated herein as reference. Thus, steviol glycosides are natural sweeteners which are significantly sweeter than glucose, and of dextrose, in very low concentrations. Thus, steviol glycosides can sweeten a composition, with minimal changes to the caloric values of the composition to the osmolarity of the composition, which is directly proportional to the amount of particles in a composition.

It is within the scope of the invention wherein a major benefit for using non-caloric sweeteners, as part of the diet, is to improve the organoleptic properties of the food in question. Thus, enabling an enhanced acceptance of both the foods and reduced caloric value, as compared with its original higher calorie version. It was stated that “Palatability. Another of the major benefits of using non-caloric sweeteners, as part of the diet is to improve the organoleptic properties of the food in question, thus enabling improved acceptance both of the foods themselves and reduce calorie meals in which any food of this type is used, compared with its original high calorie version and which contain sugar as such, which undoubtedly contributes to optimal organoleptic properties”, see García-Almeida, J. M., M. Gracia, and J. García Alemán “A current and global review of sweeteners; regulatory aspects.” Nutricion hospitalaria 28. Supl 4 (2013): 17-31; e.g., as cited from page 26 para 7, which is incorporated herein as reference. In light of the above, the use of steviol glycoside extract in the composition is made in order to improve palatability of the composition, without increasing its osmolarity or its glycemic index. Furthermore, it is prepared to be sweet and slightly acidic, in order to further increase its palatability. Inclusion of acids in food and beverage have has a few roles: Enhancing flavor of the foods and beverages; Controlling pH, for inhibition of microbial growth; and, Preserving the original characteristics of the food or beverages, as follows: “Acids are found in a wide variety of foods such as baked goods, beverages, confections, gelatin desserts, jams, jellies, dairy products, processed meats, fats, and oils. Their main use is to provide and enhance flavor of foods and beverages. In addition to contributing to flavor, acidulants are commonly used for pH control to inhibit microbial growth in food products and aid in gelling properties of gelatin desserts, jams, jellies, and jellied candies. Many acidulants also have the ability to chelate trace metal ions and act as a synergist with antioxidants). Moreover, food acids have been used to prevent nonenzymatic browning, modify viscosity and melting properties, provide leavening, and act as a curing agent”, see Da Conceicao Neta, Edith Ramos, Suzanne D. Johanningsmeier, and Roger F. McFeeters. “The chemistry and physiology of sour taste—a review.” Journal of food science 72.2 (2007) which is incorporated herein as reference.

The combination of natural sweetener steviol glycoside with the acidic taste achieved by citric acid, provides a synergistic palatable effect as follows: “Products that have some acidity are easier to work with, because acids and stevia have a nice effect together.” And “Stevia's synergistic effect in citrus-based products is evident in products such as Coke's Sprite”, see “Sweet Options”, Food product design, vol. 21, no. 2, February 2011, e.g., as cited in page 7, para Sand 5, which is incorporated herein as reference.

Sweet and umami (the taste of monosodium glutamate) are the main attractive taste modalities, and these tastes are attractive to human and animals: “Sweet and umami (the taste of monosodium glutamate) are the main attractive taste modalities”, see Li, Xiaodong, et al. “Human receptors for sweet and umami taste.” Proceedings of the National Academy of Sciences 99.7 (2002): 4692-4696, e.g., as cited in page 4692, abstract; and Shizuko Yamaguchi, and Kumiko Ninomiya. “Umami and food palatability.” The Journal of nutrition 130.4 (2000): 921S-926S, which are both incorporated herein as reference. Furthermore, monosodium glutamate (MSG) enhances palatability and intake of food, even new food”, see Bellisle, France. “Experimental studies of food choices and palatability responses in European subjects exposed to the Umami taste.” Asia Pacific journal of clinical nutrition 17.S1 (2008): 376-379, e.g., as cite in abstract, which is incorporated herein as reference.

It is hence in the scope of the invention to disclose a novel composition and method for either treating or inducing of remission of IBD, and more specifically, compositions, diets, nutraceuticals and nutrition (e.g., EN, PEN), means and methods for treating or inducing of remission of inflammation in IBD patients is disclosed, where the methods comprise, inter alia and in a non-limiting manner steps of preparing an oral rehydration solution; administering the oral rehydration solution to the individual animal; 1-glutamic acid in a range of about 0.01% to about 0.40% w/w; about 1.50% w/w glucose monohydrate; about 0.20% w/w sodium chloride; about 0.15% w/w potassium chloride; about 0.15% w/w sodium dihydrogen phosphate; about 0.10% w/w xanthan gum; hydrolyzed whey in a range of about 0.15% to about 1.00% w/w; about 85% steviol Glycoside extract I; and a range of about 0.01% to about 0.03% w/w; about 0.20% w/w citric acid monohydrate; and monosodium glutamate in a range of about 0.05% to about 0.80% w/w; and (iv) about 0.35% w/w glycine.

Oral rehydration therapy has an important place in the management of veterinary patients. The oral liquid rehydration formulation of the present invention may be used as an initial supportive treatment in any anorexic or vomiting animal and can be used alongside intravenous fluids. Oral rehydration therapy may be continued until a transition to more complex foods can be made. Oral rehydration therapy using the oral liquid rehydration formulation of the present invention allows the body to gain essential nutrients and electrolytes without burdening digestive processes. Oral liquid rehydration formulation according to this invention which are highly palatable and which are nutritious to veterinary patients are likely to be better accepted and tolerated.

The present invention hence discloses compositions, diets, top-dressing, foodstuffs, nutraceuticals and nutrition (e.g., EN, PEN), means and methods for treating or inducing of remission of inflammation in IBD patients whilst significantly reducing antibiotics usage.

The present invention also discloses compositions, diets, top-dressing, foodstuffs, nutraceuticals and nutrition (e.g., EN, PEN), means and methods for treating or inducing of remission of inflammation in IBD patients whilst significantly gaining weight.

The present invention also discloses compositions, diets, top-dressing, foodstuffs, nutraceuticals and nutrition (e.g., EN, PEN), means and methods for treating or inducing of remission of inflammation in IBD patients and administering patients with normal levels of serum immunoglobulin, improving fecal score, decreasing weight loss compared to a control group, fed with standard feed.

The present invention also discloses compositions, diets, top-dressing, foodstuffs, nutraceuticals and nutrition (e.g., EN, PEN), means and methods for treating or inducing of remission of inflammation in IBD patients by providing an improved small intestine anatomy, namely increased small intestine's villi size in the jejunum.

The present invention further discloses compositions, diets, top-dressing, foodstuffs, nutraceuticals and nutrition (e.g., EN, PEN), means and methods for treating or inducing of remission of inflammation in IBD patients delivery method for oral vaccine and antibiotics: Significant increased uptake of oral antibiotics a solution of Px was demonstrated in piglets which offered a solution of antibiotic in water or antibiotic in a 3% solution of Px, for two days, in a cross-over study type. The antibiotic used was Coliphur®, which is a mixture of neomycin and colistin. Furthermore, uptake of oral antibiotics in a solution of Px reached.

### Px Consumption

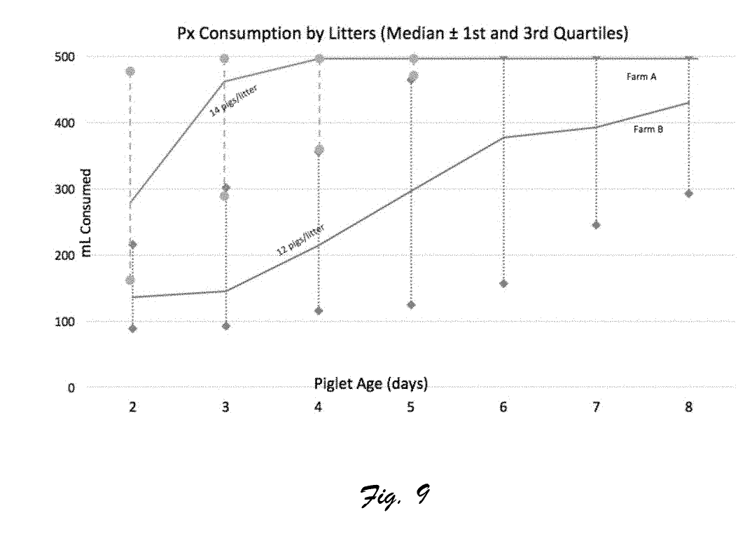

On Farm A, consumption of Px increased quickly during the first week of life. By day 3 of age, the median consumption of Px was 500 mL/litter/day. Litters contained an average of 14 piglets, with a median consumption of 36 mL/pig on day 3 of treatment, see FIG. 9. On Farm B, the consumption of Px also increased steadily during the first week of life, reaching a median consumption of 430 mL/litter on day 8. Litters contained an average of 12 piglets, with a median consumption of 18 mL/pig on day 3 of treatment.

### Meta-Analysis of Pre-Weaning Mortality

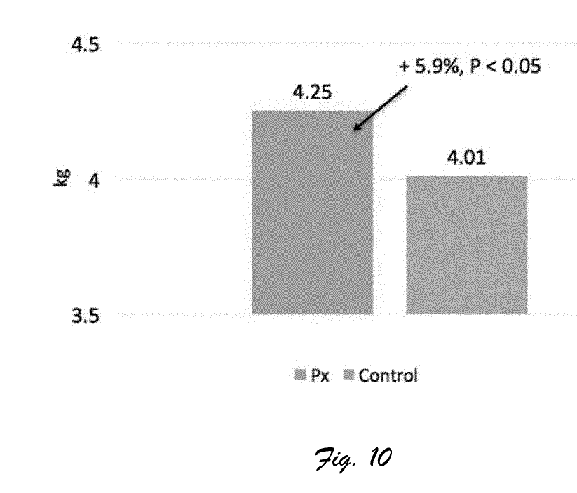

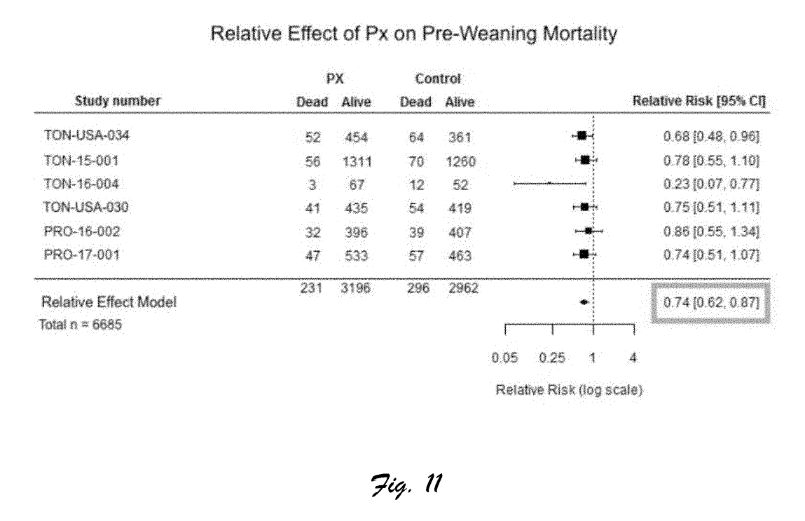

A meta-analysis of pre-weaning mortality was performed using data from 6 studies. These studies were conducted in various sites, both in Spain and the USA, over a 12-month period. All treatment litters received Px 500 mL/day in an open pan from days 2-8 of age, while control litters received no supplementation. All piglets were allowed to suckle normally. All litters were weaned at ˜21 days of age. Data was analyzed using the litter as the experimental unit. All studies for which litter mortality data was available were included. Therefore, the meta-analysis was based on 6 trials, 543 litters and 6,685 piglets. FIG. 10 is a forest plot that shows the relative risk of pre-weaning mortality in the treated piglets versus the control group with corresponding 95% confidence intervals in the individual studies. The analysis is based on a random-effects model performed with R-package Metafor. Piglets who are given Px have only a 0.74 risk of pre-weaning mortality compared to the control group (95% confidence interval 0.62-0.87, P=0.001). Reference is now made to FIG. 11 shows PWM data for the included trials. Conclusion: Px reduced pre-weaning mortality by 26%.

### Px Consumption Data

The volume of Px consumed by neonatal pigs from days 2-8 of age was measured carefully in 4 studies, comprising 278 litters. By the second day of being offered Px (day 3 of age), most litters were consuming over 400 mL of Px per day. Reference is now made to FIG. 12 that shows average daily Px consumption (aggregated data).

All references cited herein are intended to be incorporated by reference. Although the present invention has been described above in terms of specific embodiments, it is anticipated that alterations and modifications to this invention will no doubt become apparent to those skilled in the art and may be practiced within the scope and equivalents of the appended claims. The disclosed embodiments are illustrative and not restrictive, and the invention is not to be limited to the details given herein. There are many alternative ways of implementing the invention. It is therefore intended that the disclosure and following claims be interpreted as covering all such alterations and modifications as fall within the true spirit and scope of the invention.

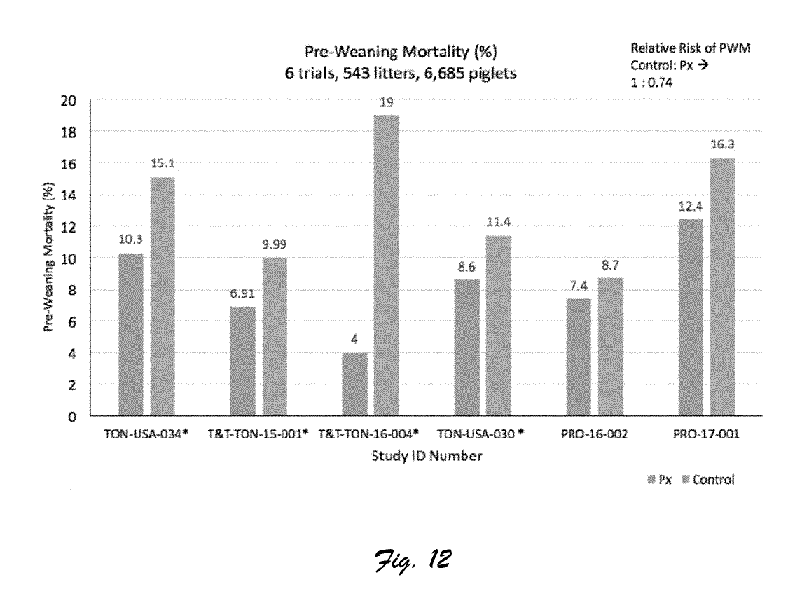

FIG. 13 depicts summary of study design. The pre- and post-weaning feeding plan is summarized in Table 8. Four days before weaning (approximately 15 days of age, study day −4), all pigs were weighed and litters were allocated to one of 3 new treatment groups—dry creep feed (D), water—gruel (WG) or Px—gruel (PG). Group A and B pigs were allocated equally across the 3 new treatment groups. These new groups were again balanced by sow's parity, number of piglets and piglets' weight on SD −4. On SD −4, D litters received dry creep feed, WG litters received 500 mL of water and PG litters received 500 mL of 3% Px solution. All feeds and liquids were served in an open pan. On SD −3, SD −2, and SD −1, D litters continued to receive dry creep feed. WG litters received a gruel of creep feed mixed with water and PG litters received a gruel of creep feed mixed with Px solution. All gruel was made using the ratio of 10 kg of dry feed to 15 L of liquid (water or 3% Px solution), i.e., a 1:1.5 w/v ratio. On SD 0 (19 days of age), pigs were weaned, weighed and sorted into 53 pens of 10-11 pigs each. Pigs were also sorted into pens by bodyweight (heavy (H), medium (M), light (L)) while still remaining within their feed groups. Heavy was defined as >5.8 kg, medium 4.1-5.8 kg and light <4.1 kg. These weights corresponded to the upper 25% of pigs, middle 50% of pigs, and lower 25% of pigs. WG and PG pigs received their gruel on the day of weaning, while D pigs received dry creep feed. Both WG and PG pigs continued to receive their gruel ad libitum for at least 1 day after weaning and were then tapered off gruel over another 3-4 days according to body weight. Light and medium pigs were tapered more slowly. All pigs also had dry creep feed available ad lib in separate feeders. Gruel was fed twice daily.

### Dry Matter Intake

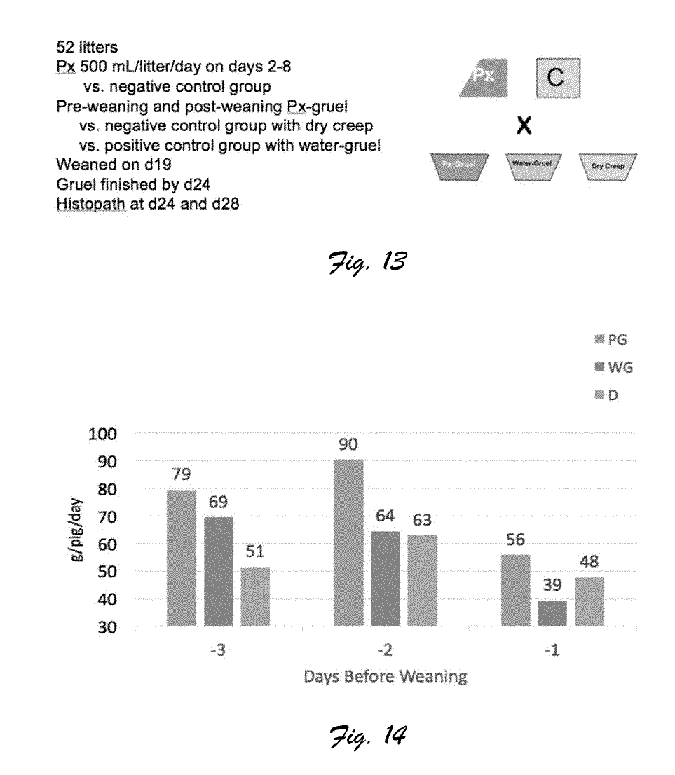

In the pre-weaning period, pigs who received Px-gruel had the greatest DMI as shown in FIG. 8. FIG. 14 depicts Daily DMI from 1 to 3 Days before Weaning. The Px-gruel group had an average DMI of 54±3.6 g/kg BW in the pre-weaning period, which was significantly higher than the water-gruel group (40±3.5 g/kg BW) or the dry creep group (39±4 g/kg BW) (P<0.05). This pattern of consumption continued over the next 6 days post-weaning as shown below. Reference is made to FIG. 15 which is presenting Daily DMI intake for 6 days Post-Weaning (g/pig/day).

The DMI was also tracked for the pens of Heavy, Medium and Light pigs. The table below shows total DMI of gruel and creep feed/kg BW in the first week and second weeks after weaning. The most marked differences were seen in the Light and Medium pigs in the first week after weaning. Both gruel groups had significantly higher DMI compared to the dry feed group.

1. A composition for either treating IBD or inducing of remission of IBD patients, characterized by that that said composition consists at least two member of a group consisting of steviol glycoside, citric acid monohydrate, monosodium glutamate and glycine.

2. The composition for either treating IBD or inducing of remission of IBD patients of claim 1, characterized by that that said composition consists at least three member of a group consisting of steviol glycoside, citric acid monohydrate, monosodium glutamate and glycine.

3. The composition for either treating IBD or inducing of remission of IBD patients of claim 1, characterized by that that said composition consisting of steviol glycoside, citric acid monohydrate, monosodium glutamate and glycine.

4. The composition for either treating IBD or inducing of remission of IBD patients of claim 1, wherein the compositions are (i) about 85% steviol glycoside extract ranging from about 0.01% to about 0.03% w/w, (ii) about 0.20% w/w citric acid monohydrate; (iii) monosodium glutamate ranging from about 0.05% to about 0.80% w/w; and (iv) about 0.35% w/w glycine.

5. The composition for either treating IBD or inducing of remission of IBD patients of claim 1, wherein at least one of the following is held true: (i) said composition comprises 1-glutamic acid in a range of about 0.01% to about 0.40% w/w and monosodium glutamate in a range of about 0.05% to about 0.80% w/w; (ii) said composition comprises about 1.50% w/w glucose monohydrate; (iii) said composition comprises about 0.20% w/w sodium chloride; (iv) said composition comprises about 0.15% w/w potassium chloride; (v) said composition comprises about 0.15% w/w sodium dihydrogen phosphate; (vi) said composition comprises about 0.10% w/w xanthan gum; (vii) said composition comprises about 0.35% w/w glycine; (viii) said composition comprises about 0.30% w/w trisodiumcitrate; (ix) said composition comprises about 0.20% w/w citric acid monohydrate; (x) said composition comprises 85% Steviol Glycoside extract in a range of about 0.01% to about 0.03% w/w; (xi) wherein said composition comprises hydrolyzed whey in a range of about 0.15% to about 1.00% w/w; (xii) said composition comprises about 1.00% w/w hydrolyzed wheat; (xiii) said composition comprises cereals as a protein source.

6. The composition for either treating IBD or inducing of remission of IBD patients of claim 1, wherein said composition further comprises at least one or more members of a group consisting of 1-glutamic acid in a range of about 0.01% to about 0.40% w/w; about 1.50% w/w glucose monohydrate; about 0.20% w/w sodium chloride; about 0.15% w/w potassium chloride; about 0.15% w/w sodium dihydrogen phosphate; about 0.10% w/w xanthan gum; and hydrolyzed whey in a range of about 0.15% to about 1.00% w/w.
The composition is tailored to suit the flavouring preferences of different species or individuals.

7. The composition for either treating IBD or inducing of remission of IBD patients of claim 1, provided as defined in any of examples 1 to 6.

8. The composition for either treating IBD or inducing of remission of IBD patients of claim 1, wherein said composition is an isotonic, hypotonic or hypertonic solution.

9. The composition for either treating IBD or inducing of remission of IBD patients of claim 1, wherein said composition is provided in one member of a group consisting of diets, foodstuffs, edibles beverages, top-dressings, food dressings, food additives, drugs, medicaments, pharmaceuticals, nutraceuticals and nutrition, including EN and PEN, carriers for nutraceuticals, medicament (including oral vaccine & antibiotics) & flavor enhancers [NMFs] prebiotics and toppers.

10. A method for either treating IBD or inducing of remission of IBD patients, characterized by administering a composition consists at least two member of a group consisting of steviol glycoside, citric acid monohydrate, monosodium glutamate and glycine.

11. The method of claim 10, characterized by administering a composition consists at least three member of a group consisting of steviol glycoside, citric acid monohydrate, monosodium glutamate and glycine.

12. The method of claim 10, characterized by administering composition consisting of steviol glycoside, citric acid monohydrate, monosodium glutamate and glycine.

13. The method of claim 10, characterized by administering a composition comprising (i) about 85% steviol glycoside extract ranging from about 0.01% to about 0.03% w/w, (ii) about 0.20% w/w citric acid monohydrate; (iii) monosodium glutamate ranging from about 0.05% to about 0.80% w/w; and (iv) about 0.35% w/w glycine.

14. The method of claim 10, wherein at least one of the following is held true: (i) said composition comprises 1-glutamic acid in a range of about 0.01% to about 0.40% w/w and monosodium glutamate in a range of about 0.05% to about 0.80% w/w; (ii) said composition comprises about 1.50% w/w glucose monohydrate; (iii) said composition comprises about 0.20% w/w sodium chloride; (iv) said composition comprises about 0.15% w/w potassium chloride; (v) said composition comprises about 0.15% w/w sodium dihydrogen phosphate; (vi) said composition comprises about 0.10% w/w xanthan gum; (vii) said composition comprises about 0.35% w/w glycine; (viii) said composition comprises about 0.30% w/w trisodiumcitrate; (ix) said composition comprises about 0.20% w/w citric acid monohydrate; (x) said composition comprises 85% Steviol Glycoside extract in a range of about 0.01% to about 0.03% w/w; (xi) wherein said composition comprises hydrolyzed whey in a range of about 0.15% to about 1.00% w/w; (xii) said composition comprises about 1.00% w/w hydrolyzed wheat; (xiii) said composition comprises cereals as a protein source.

15. A method for providing an oral rehydration therapy, improving small intestine anatomy & increasing small intestine's villi size weight gaining, weight gaining associated with reduction in antibiotics usage, for improving weight gain whilst decreasing use of milk replacers, improving small intestine anatomy & increasing small intestine's villi size, or increasing the uptake of nutraceuticals, medicament (including oral vaccine & antibiotics) & flavour enhancers uptake, comprising step of administering a patient a composition defined in any of claim 1, 2 or 3.
